(19) United States
(12) Patent Application Publication
(10) Pub. No.: US 2025/0303128 A1
(43) Pub. Date: Oct. 2, 2025

(54) SHUNT FOR USE IN DISORDERS OF FLUID ACCUMULATION

(71) Applicant: The Board of Regents of the University of Texas System, Austin, TX (US)

(72) Inventors: Lovepreet Mann, Austin, TX (US); Ramesha Papanna, Austin, TX (US)

(21) Appl. No.: 19/097,692

(22) Filed: Apr. 1, 2025

Related U.S. Application Data
(60) Provisional application No. 63/573,438, filed on Apr. 2, 2024.

(51) Int. Cl. A61M 27/00

(52) U.S. Cl. CPC A61M 27/008; A61M 2027/004

(57) ABSTRACT

Disclosed is a device for intrauterine shunting of fluid accumulation in organs. The barb at the proximal end of the disclosed shunt helps anchor the shunt in place, easing its placement and decreasing its dislodgement in-vivo, while the pigtail loop at the distal end maintains the shunt in place within an organ of a fetus. The stiffer shunt prevents occlusion from kinking and external pressure. Both of these properties may have a significant impact on shunt surgery and outcomes.

### TECHNICAL FIELD

Provided herein is a device for organ fluid shunting, e.g., intrauterine shunting of fluid accumulation in fetal organs, e.g., gastrointestinal (GI), or e.g., urology shunting. Fetal shunts are flexible tubes, generally made of plastic, that are placed in utero under continuous ultrasound monitoring, guided percutaneously to a fetal space that has fluid accumulation.

### SUMMARY

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The present disclosure describes improved shunts for use in organ fluid shunting procedures, e.g., intrauterine shunting of fluid accumulation in fetal organs, e.g., gastrointestinal (GI), or e.g., urology shunting. The barb at the proximal end facilitates the anchoring of the shunt in place, easing the placement and decreasing the dislodgement, while the pigtail loop at the distal end facilitates the maintenance of the shunt in place. The stiffer shunt is designed to aid in preventing occlusion from kinking and external pressure. Both of these properties provide a significant improvement on organ surgery, e.g., fetal surgery and outcomes. In many configurations, the proximal pigtail of a device of the disclosure is made of softer material, which, when implanted into a subject, prevents organ damage (i.e., unintentional puncture). In such configurations, the distal pigtail remains stiffer to reduce dislodgement into the body.

In some aspects, provided herein is a shunt comprising: a hollow tube, wherein the hollow tube comprises two ends and an intermediary section, wherein the two ends are curved pigtails, and wherein one or more barbed ends are positioned between the intermediary section and at least one of the curved pigtails. In some instances, the intermediary section has a length of between 2.5 cm to 4.5 cm. In some instances, each of the two curved pigtails comprise at least one 360 degree full loop. In other instances, at least one of the two curved pigtails comprise one and half loops. In the shunts/shunts of the disclosure, the two ends are curved pigtails with a certain orientation with respect to each other. In some instances, one of the two curved pigtails has a clockwise orientation and the other has a counterclockwise orientation. In some instances, each of the two curved pigtails have a same orientation, either clockwise or counterclockwise. In preferred embodiments, the shunt is a fetal shunt, a GI shunt, or a urology shunt. In some configurations of the device, one of the two curved pigtails (e.g., at the proximal end, the distal end, or both) of the shunt/shunt is softer with respect to the rigidity of the intermediary segment. In some configurations, a tensile strength of one or both of the two curved pigtails is significantly softer than a tensile strength of the remaining of the shunt. In some configurations, said intermediary section does not have any perforations. In some configurations, the hollow tube has an unstretched length of about 2 cm to 7 cm, an unstretched length no longer than 7 cm, or an unstretched length of about 4 cm. In some configurations, each of the two curved pigtails has a plurality of perforations. In some configurations, the plurality of perforations in at least one of the two curved pigtails is no more than 15 perforations or no more than 10 perforations. In other configurations, the plurality of perforations in both of the two curved pigtails is no more than 15 perforations or no more than 10 perforations. The shunt can be manufactured with certain dimensions, e.g., in some configurations the outer diameter of the hollow tube can be approximately 1 millimeter (mm) and its circumference is approximately 3.14 mm, the outer diameter of the hollow tube can be approximately 1.33 mm and its circumference is approximately 4.19 mm, the outer diameter of the hollow tube can be approximately 1.67 mm and its circumference is approximately 5.24 mm, or the outer diameter of the hollow tube can be approximately 2.00 mm and its circumference is approximately 6.28 mm. In some configurations, one or more barbed ends are positioned between the intermediary section and one of the curved pigtails are two barbed ends, thus defining a proximal end of the shunt. Other configurations may comprise two barbed ends symmetrically placed between the intermediary section and both of the curved pigtails. In many instances, the one or more barbed ends comprise at least two barbed ends placed between the intermediary section and one or both of the curved pigtails.

In some aspects the disclosure describes a shunt, e.g., fetal, GI, or urology comprising: a hollow tube having an intermediary section; a curved pigtail at a distal end of the hollow tube, wherein said distal curved pigtail comprises a plurality of perforations, said distal curved pigtail perpendicularly positioned with respect to said intermediary section; a curved pigtail at a proximal end of the hollow tube, wherein said proximal curved pigtail comprises a plurality of perforations, said proximal curved pigtail perpendicularly positioned with respect to said intermediary section; a barbed end placed between the intermediary section and the proximal end.

In some aspects, the disclosure describes a shunt comprising: a hollow tube having an intermediary section; a curved pigtail at a distal end of the hollow tube, wherein said distal curved pigtail comprises a plurality of perforations, said distal curved pigtail perpendicularly positioned with respect to said intermediary section; a curved pigtail at a proximal end of the hollow tube, wherein said proximal curved pigtail comprises a plurality of perforations, said proximal curved pigtail perpendicularly positioned with respect to said intermediary section; and one or more barbed ends placed between the intermediary section and the proximal end.

In some aspects, the disclosure provides a method for treating a subject with excessive fluid in a region of its body, the method comprising implanting into said subject a shunt comprising a hollow tube, wherein the hollow tube comprises two ends and an intermediary section, wherein the two ends are curved pigtails, and wherein one or more barbed ends are positioned between the intermediary section and at least one of the curved pigtails. In some aspects, the region of the subject body is a bladder or a thoracic cavity. In some instances, the implanting occurs on a fetal subject at any period of its gestational time.

In some instances, a device of the disclosure is manufactured with three-dimensional (3D) printing. In particular instances, a stent/shunt described herein is manufactured with fused deposition modeling (FDM), which uses a continuous filament of a thermoplastic material.

### Terminology

Unless defined otherwise, all technical and scientific terms used herein have the same meaning as commonly understood by one of ordinary skill in the art to which this invention belongs. For purposes of interpreting this specification, the following definitions will apply and whenever appropriate, terms used in the singular will also include the plural and vice versa.

As used herein, the term “pigtail” is used to describe a structure at the end of a shunt that comprises at least one “full-coil,” a “full spiral,” or “series of loops” with at least one loop of at least 180, 270, or 280 degrees, on the end of a medical device, such as a shunt, used to hold the device in place within a hollow organ or chamber. The term pigtail encompasses complete loops. The term pigtail does not encompass partial loops, i.e., loops that are shorter than 180 degrees. The term pigtail encompasses complete one complete loop (i.e., at least 180, 270, or 280 degree loop) and any additional turns (i.e., one complete full loop and a partial loop is considered a pigtail for the purposes of this definition).

As used herein, the term “French gauge” (Fr) or “French gauge system” is used in reference to the standard system for catheter sizes. As used herein, and as conventionally used, one increment on the French scale is equal to ⅓ millimeter, e.g. 8 Fr catheter is 8×0.33 mm=2.67 mm in caliber. French sizes with equivalent metric diameter and circumferences contemplated in the instant disclosure comprise: 3 Fr is 1 mm (diameter) and 3.14 mm (circumference); 4 Fr is 1.33 mm and 4.19 mm; 5 Fr is 1.67 mm and 5.24 mm; 6 Fr is 2 mm and 6.28 mm; 7 Fr is 2.33 mm and 7.33 mm; 8 Fr is 2.67 mm and 8.34 mm; 9 Fr is 3 mm and 9.42 mm; 10 Fr is 3.33 mm and 10.47 mm; 12 Fr is 4 mm and 12.57 mm; 14 Fr is 4.67 mm and 14.66 mm; 16 Fr is 5.33 mm and 16.76 mm; and 20 Fr is 6.66 mm and 20.94 mm.

As used herein, the terms “shunt” and “stent” are used interchangeably.

As used here, the term “softer” refers to a tensile strength that is lower than a reference point. For instance, when referring to a section of a hollow tube, e.g., a softer pigtail in the proximal end of a shunt of the disclosure has a significantly lower tensile strength compared to another area of the same tube. Similarly, the term “firmer” or “more rigid”” refers to a tensile strength that is lower than a reference point.

As used herein, terms such as “first,” “second,” “third,” “fourth,” “fifth,” “sixth,” “seventh,” “eight,” “ninth,” “tenth,” “nth,” or the like merely identify one of a number of barbs, pigcoils, loops, and/or points of reference as disclosed herein, and likewise do not necessarily limit embodiments of the present disclosure to any particular configuration or orientation. Furthermore, terms such as “preceding,” “subsequent,” “left,” “right,” “top,” “bottom,” “front,” “rear,” “side,” “height,” “length,” “width,” “upper,” “lower,” “interior,” “exterior,” “inner,” and “outer” that may be used herein merely describe points of reference, e.g., for barb(s), pigcoils, or loops, and do not necessarily limit embodiments of the present disclosure to any particular orientation or configuration.

As used herein, “about” and the term “approximately,” means the recited quantity exactly and small variations within a limited range encompassing plus or minus 10% of the recited quantity. In other words, the limited range encompassed can include ±10%, ±9%, ±8%, ±7%, ±6%, ±5%, ±4%, ±3%, ±2%, ±1%, ±0.5%, ±0.2%, ±0.1%, ±0.05%, or smaller, as well as the recited value itself Thus, by way of example, “about 10” should be understood to mean “10” and a range no larger than “9-11.” For clarity, as used herein, designation of a range of values includes all integers within or defining the range, and all subranges defined by integers within the range.

As used herein, the term “and/or” refers to and encompasses any and all possible combinations of one or more of the associated listed items, as well as the lack of combinations when interpreted in the alternative (“or”).

As used herein, term “or” refers to any one member of a particular list and also includes any combination of members of that list.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments wherein any one or more of the recited elements, species, or embodiments, may be excluded from such categories or embodiments, for example, for use in an explicit negative limitation. For example, where the disclosure describes a “fetal shunt” or a “hollow tube” or an “intermediary region” with no perforations, this is intended to provide antecedent basis for a negative limitation on a region or section of the medical device.

As used herein, the singular forms of the articles “a,” “an,” and “the” include plural references unless the context clearly dictates otherwise. For example, the term “a protein” or “at least one protein” can include a plurality of proteins, including mixtures thereof.

As used herein, the term “subject,” refers to a human, including an unborn fetus for which the described shunts are intended to be used. It is specifically contemplated that a fetal shunt of the disclosure may also be used with a premature infant (an infant born prior to complete 40 weeks of gestational time). The term subject also encompasses newborn humans, and humans of other ages who can benefit from drainage of liquid in a region of their body's.

Statistically significant means p≤0.05.

### DESCRIPTION OF DRAWINGS

The patent or application file contains at least one drawing executed in color. Copies of this patent or patent application publication with color drawing(s) will be provided to the Office upon request and payment of the necessary fee.

The foregoing and other features and advantages of the present invention will be more fully understood from the following detailed description of illustrative embodiments taken in conjunction with the accompanying drawings in which:

FIGS. 1A-B (FIGS. 1A-B) shows a configuration of a fetal shunt of the disclosure. Illustrated in FIG. 1A is a curved pigtail at a distal end of the hollow tube 101, an intermediary segment at a 90 degree angle from said distal pigtail 102, 103 a curved pigtail at a proximal end of the hollow tube, and two barbed ends 104 placed between the intermediary segment and the proximal end. FIG. 1B is a shunt incorporating the barbs in mid segment, and double loop on both sides with 90 degree angles. It was created using a heat mold technique.

FIG. 2 (FIG. 2) is an illustration of a shunt of the art consisting of extended coils present in the double curved pigtails. FIG. 2 also underscores the absence of any barb on this device.

FIGS. 3A-E (FIGS. 3A-E) are illustrations of shunts of the art consisting of a same manufacturer. The figures underscore perforation(s) in the intermediary segment of these device, as well as a flat (same plane) design of a partial coil.

FIG. 4 (FIG. 4) is an illustration of a shunt of the art consisting of a nitinol wire mesh.

FIGS. 5 and 6 (FIGS. 5-6) are illustrations of shunts of the art from a same manufacturer. The figures underscore perforation(s) in the intermediary segment of these device, as well as a flat (same plane) design and variations of single loops at the end (partial coiled loop FIG. 5; pigtail loop FIG. 6).

FIG. 7 (FIG. 7) is an illustration of a shunt of the art consisting of a single curved loop at one end and double barbed wires at the other end.

FIG. 8 (FIG. 8) is an illustration of a shunt of the art having reinforced retention structures.

FIGS. 9A-D (FIGS. 9A-D) are illustrations of a transcutaneous ultrasound of rat thorax (FIG. 9A), results of the weight challenge of two structurally distinct stents in a rat chest wall (FIGS. 9B-C), and a chart quantitating the dislodgement weight (D).

FIGS. 10A-D (FIGS. 10A-D) are color pictures showing a comparison of the stent entry site of stents in a rat model. FIG. 10A depicts the stent entry site of a stent with a design shown in FIG. 2. FIG. 10B depicts the functional utility of the barb of a stent with a design shown in FIG. 2 in preventing dislodgement. FIG. 10C depicts the stent entry site of a stent with a design shown in FIG. 3a of size 5Fr.

FIGS. 11A-F (FIGS. 11A-F) are color pictures showing a stent of FIG. 3A in a human fetus application. FIG. 11A is a color picture of an ultrasound of a 14 weeks 6 days fetus with lower urinary track obstruction (LUTO) with “key hole” sign with oligohydramnios and chest compression. FIG. 11B is a color picture of an ultrasound of the fetus at 22 weeks following a “dislodged” shunts (FIG. 2 stent design)×2 with oligohydramnios. FIG. 11C is a color picture of an ultrasound of a fetus at 22 weeks with bilateral hydroutereter and hydronephrosis. FIG. 11D is a color picture of an ultrasound of a fetus at 22 weeks 3 days during the placement of a stent (FIG. 3D stent design) (5Fr) 5 cm length in the bladder. Cook needle and introducer noted through the maternal abdominal wall and uterus (blue arrows), and stent barbed (yellow arrow) and pigtail noted outside the fetal abdomen. FIG. 11E is a color picture of an ultrasound of the fetus at 30 weeks showing the stent in place through the bladder (white arrow) and piercing through the bladder dome into the peritoneal space with collection of fluid (red arrow). The advantages of the pig tail and barb combination in holding the stent in place are visible. It is specifically contemplated by the disclosure that the double pig tail design could improve the odds of potential dislodgement.

It should be understood that the drawings are not necessarily to scale (e.g., schematics), and that like reference numbers refer to like features.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

Consider, for instance that worldwide, the large majority of fetal shunting operations are performed using double-pigtail catheters. The two most common medical devices for treating these conditions are vesicoamniotic shunts for fetal bladder outlet obstruction and thoracoamniotic shunts for fetal hydrothorax.

Vesicoamniotic shunts are generally used for treating urinary tract obstruction that occurs in utero. Such obstructions can block the flow of fetal urine, which makes up amniotic fluid. Such blockages can have serious consequences for the fetus. Without adequate levels of amniotic fluid, a fetus may not develop properly, with the fetal lungs being especially affected. Fetal urinary tract obstructions can range from mild to severe. Treatment with current shunts generally involve the insertion of a small hollow tube, called a vesicoamniotic shunt, through the mother's abdomen and into the fetus's bladder. The procedure is similar to amniocentesis; however, in a vesicoamniotic shunt the catheter is left in the bladder, where it allows urine to flow out of this organ. While the technique can be effective, commercially available shunts have a tendency to dislodge over time, requiring repeat procedures in about half of the cases, or can clog. It is speculated that the very small channel through which urine must flow plays a role on the dislodgement of the existing shunt. Repeated procedures can increase the risk for complications, including premature birth.

A similar shunt, a thoracoamniotic shunt, is the treatment of choice for management of the fetus with symptomatic fetal hydrothorax (FHT). Fetal hydrothorax (FHT) is a heterogeneous condition occurring in 1:10,000-15,000 pregnancies when abnormal amounts of fluid form within the chest of a fetus. This fluid may be in the space between the lungs and the chest wall (pleural space) or within the core of the lung or chest masses. Fetal hydrothorax may also be referred to as a pleural effusion. Because the chest is an enclosed space, the presence of fluid can compress the lungs and even displace the heart.

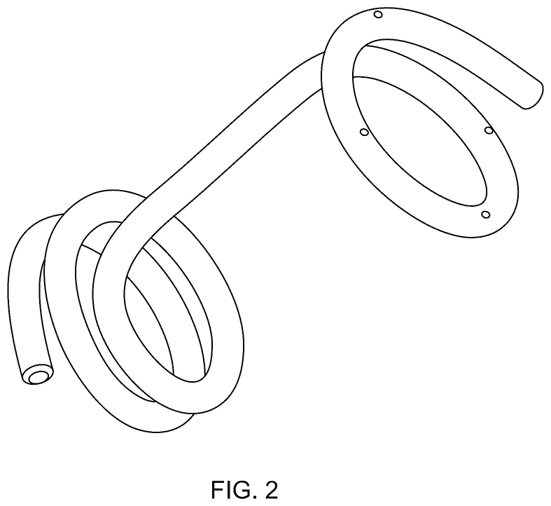

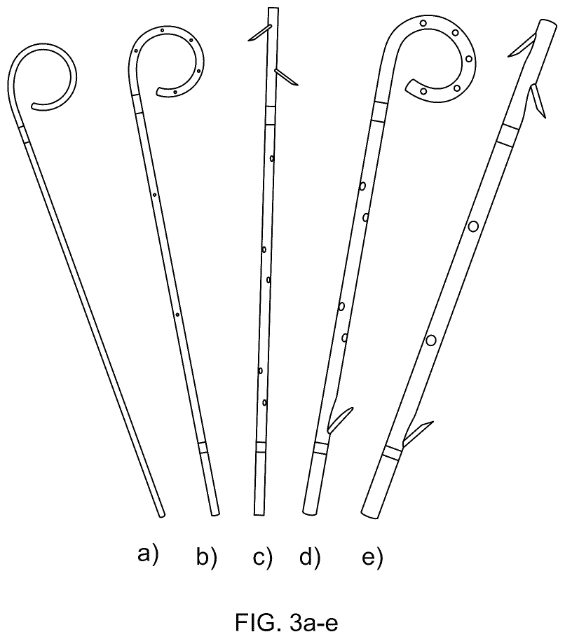

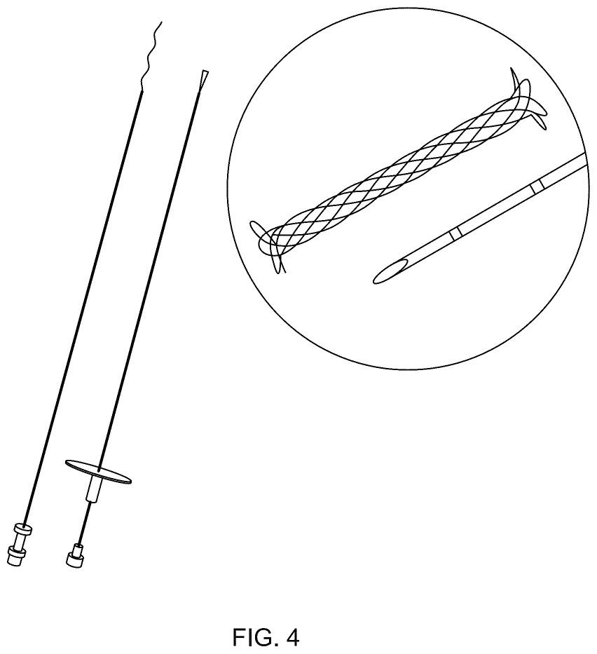

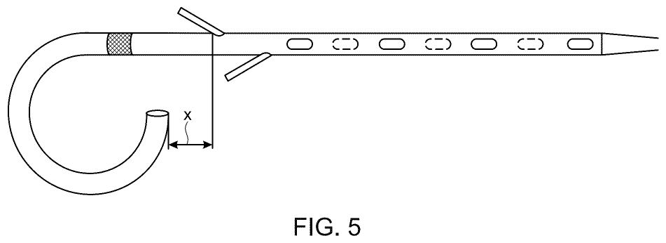

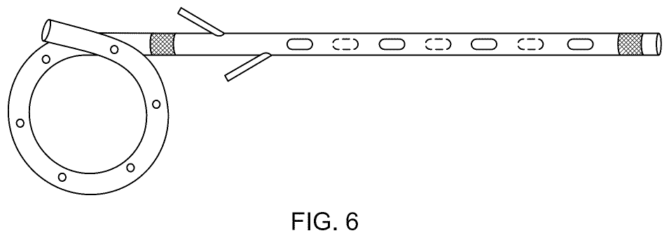

Several shortcomings have been observed with existing fetal shunt designs. In the United States, the Harrison Fetal Bladder stent is the only FDA-approved option for fetal shunting. The Harrison fetal bladder stent (Cook Medical Inc., Bloomington, IN, USA) is a double pigtail stent with an outer diameter of 5 Fr (1.67 mm) and inner diameter of 0.97 mm (See FIG. 2). The usable length between the pigtails is approximately 5-35 mm; and it is typically introduced into patients through a 13 gauge (G) needle. However, there are several shortcomings to this sole US FDA approved market option. First, the Harrison Fetal Bladder stent is difficult to place during a procedure due to its pigtail design providing to be insufficient for maintaining the product in place without dislodgement. Second, the perforations at the end of the pigtails have proved to be ineffective in draining liquid. The Advanix™ Pancreatic Stent (Boston Scientific™) is available with different, shorter, coils and/or barb ends (See FIGS. 3A-E). It can be used with endoscopic and radiopaque markers for enhanced visualization. One shortcoming of the Advanix™ series of devices, however, have proven to be the perforation(s) in the intermediary segment. The other shortcoming has been the flat design of its shorter coil. The Somatex® intrauterine shunt (Somatex Medical Technologies GmbH, Berlin, Germany) is 25 mm long with a diameter of 2.6 mm for the expanded shunt, consisting of a nitinol wire mesh and internal impermeable silicone coating. The shunt has self-deploying parasols at both ends and can be placed through an 18 G puncture cannula (See FIG. 3). In contrast to the pigtail solutions, the small parasols are hard to grasp. The Somatex intrauterine shunt was available in Germany circa 2014. The rocket KCH™ Fetal Bladder Drain (Rocket Medical, PLC) is believed to have been designed as a competitor to the Harrison Fetal Bladder Stent, due to its similarity in design (See FIG. 4). On this model, 6 holed catheters exist with opposing coils (similar to what is shown in FIG. 2). In addition, austenitic (non-magnetic) 316-316L stainless steel rings are inserted under pressure within each distal lumen of the tube material. A non-expandable stent described in U.S. Pat. No. 8,603,185 (Cook Medical Technologies LLC and University of Colorado) discloses a non-extensible shunt comprises a proximal portion having a curved portion configured for placement proximal to a sphincter and a distal portion having retaining member extending outward from a proximal end of the distal portion (See FIGS. 5 & 6). As shown in the figures, these stents require perforations in the intermediate regions.

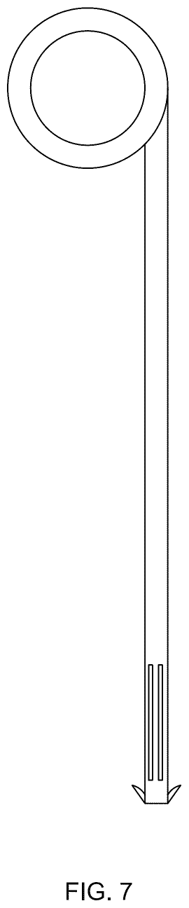

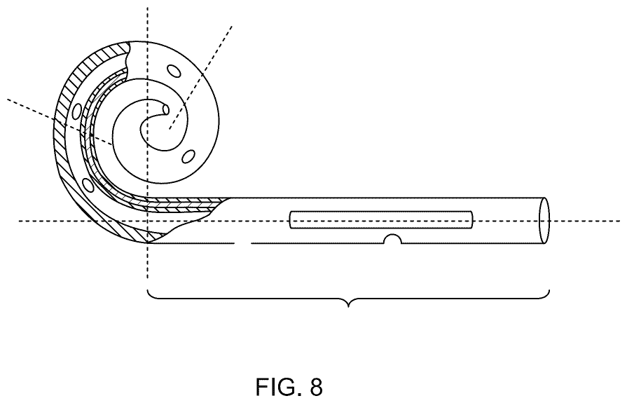

Substantially different geometries and shunt configurations have been disclosed in the art. U.S. Pat. No. 8,167,927 (Trivascular Inc, Endologix LLC) discloses a shunt with a radially self-expanded metallic shunt, including a serpentine configuration having a plurality of struts and having a plurality of proximal and distal apices. A barb integrally formed as an extension of each strut and extending outwardly from a position on a strut. Somewhat similar to the struts, U.S. Pat. No. 10,959,864 (Cook Medical Technologies, LLC) discloses a shunt with ring structures made up of at least one wire and at least one tubular connector through which a portion of the wire is disposed. The tubular connector includes a side wall with aperture formed therethrough. A portion of the wire extends therethrough and acts as a barb. Shunts with crush-resistant zones have also been described in the art, but proved impractical for use in fetal applications. U.S. Pat. No. 10,357,386 describes a prothesis system for branched body lumen. Some anti-reflux ureteral shunts share certain characteristics with the Advanix™ Pancreatic Stent, such as one curved pigtail at the distal end, e.g., U.S. Pat. No. 8,142,386 (Boston Scientific Scimed Inc.). Such stents may include a valve near the proximal end and distal to the second retention structure. The valve may include one or more slits or windows tending to render the elongated member collapsible (See FIG. 7). To counter this, some models incorporate a reinforced retention structure, e.g., U.S. Pat. No. 7,169,139 (Boston Scientific Scimed Inc.).

None of the aforementioned models, however, have addressed unique characteristic and requirements for effectively draining substances from organs or abscessed areas within a body of a fetus without suffering from a significant risk of dislodgement, collapse, or another failure.

The present disclosure describes novel stents/shunts for use, e.g., in human fetal bladder shunting and/or thoracic fetal shunting. The stents and shunts described herein have several distinguishing characteristics: first, the presently described stents do not contain any perforations on its intermediary segment. Second, the presently described stents comprise two pigtails protruding from a common intermediary hollow tube at 90 degree angle(s) (perpendicular geometric planes), a first pigtail located at a proximal end and a second pigtail located at the distal end. Third, between the proximal terminus of the hollow tube and the beginning of the proximal pigtail, the presently described stents comprise at least one, at least two, or at least three barbs to help anchor the stent in place, easing placement and decreasing its dislodgement. Fourth, in some configurations, the presently described stents comprise a softer pigtail at the proximal end or the distal end of the stent, which reduces the burden of tissue injury from a stent having a stiff tip (e.g., organ perforation). The disclosure contemplates that the softer pigtails better accommodate the contours of the bladder and chest cavity with lungs, thereby minimizing unintended damage from placement of the shunts/stents. Fifth, in some configurations, the presently described stents weigh up to four times less than other available market options.

Patients undergoing stent related procedures, e.g., Stent-Related Endoscopic Retrograde Cholangiopancreatography (ERCP) are at risk for various stent-related adverse events. Common complications include organ wall perforation, stent migration, stent occlusion, cholangitis, and injury to an organ, e.g., biliary or pancreatic duct.

The proposed stent design aims to mitigate two of the most common complications:

### (A) Stent Migration

Unintended migration is reported in a high number of cases, e.g., it is approximately 5% to 6% of cases of biliary or pancreatic stents. Internal migration can lead to serious complications, including jaundice, cholangitis, pancreatitis, or organ, e.g., bowel/abdomen perforation. Retrieval of proximally migrated stents is necessary and may involve various techniques such as stent retrieval devices, forceps, snares, or retrieval balloons.

### (B) Organ Wall Perforation

Organ perforation, although rare, can occur due to proximal stent migration where the stent's pointed tip exerts pressure against an organ, e.g., an abdomen or a the bowel lumen. Both plastic and metal stents have been implicated in such perforations, with no definitive data indicating higher risk associated with either material. Management of stent-induced perforation typically involves endoscopic retrieval of the stent and endoscopic closure if the patient shows no clinical signs of peritonitis. Surgical intervention is recommended in cases presenting with peritonitis or retroperitoneal fluid collections.

The present disclosure address the shortcomings in the art. The disclosure describes a stent design which incorporate features intended to reduce the risk of migration and perforation. Key design elements include:
A 90-degree angle with soft looped ends to minimize direct pressure against the bowel wall, thereby reducing the risk of perforation and facilitating safer stent placement.
A mid-segment anchoring barb that enhances ductal fixation, decreasing the likelihood of stent migration within the biliary or pancreatic ducts.

These features collectively improve the safety profile of the stent during an implantation procedure, e.g., ERCP by reducing two of the most significant complications-bowel wall perforation and stent migration. Stent(s)

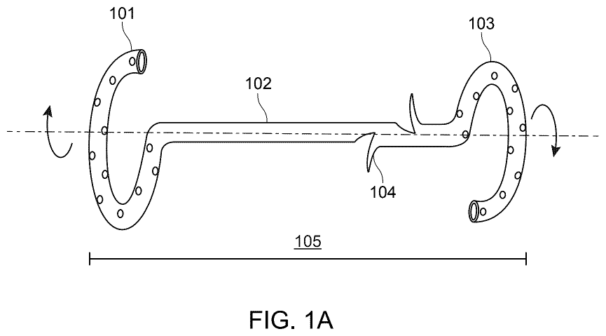

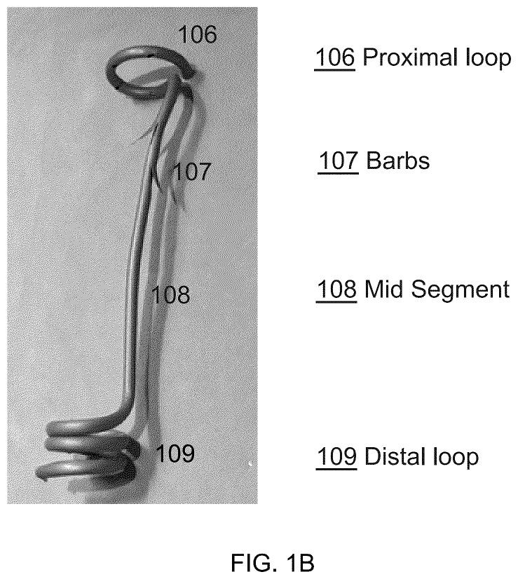

The present disclosure describes stent(s) having two pigtail loops at right degree angles, one at the proximal end 103 and the other at its distal end 102 (FIG. 1A). In the described configurations, the shunt/stent also comprises at least one 104, at least two, at least three, or another suitable number of barb ends at the proximal end, located between the intermediary segment 103 and the proximal pigtail 101. In many implementations, the size perforation at the barb end of the hollow tube is configured for implementation with a catheter that ranges from 3 Fr to 7 Fr in size.

In some cases, the disclosure provides a stent comprising: a hollow tube having an intermediary section, wherein said intermediary section does not have any perforations; a curved pigtail at a distal end of the hollow tube, wherein said distal curved pigtail comprises a plurality of perforations, said distal curved pigtail perpendicularly positioned with respect to said intermediary section; a curved pigtail at a proximal end of the hollow tube, wherein said proximal curved pigtail comprises a plurality of perforations, said proximal curved pigtail perpendicularly positioned with respect to said intermediary section; a barbed end placed between the intermediary section and the proximal end.

In some cases, the stents/shunts described herein comprising a single hollow tube. The single hollow tube can be manufactured with a certain extended length. The length of the tube can be described in terms of its “extended” length, i.e., length of the hollow tube prior to being shaped with at least two pigcoils. A length of the shunt itself can also be described with regards to the final length of an undistorted, unstretched, shunt (105). In most configurations, the shunt can be manufactured with different lengths. A shunt of an appropriate site is selected based on the size of the space in which the shunt is to be inserted, e.g., thoracic fetal shunting or fetal bladder shunting.

In some configurations, a shunt of the disclosure has an undistorted, unstretched, length (105) of 20±1 millimeters, 30±1 millimeters, 40±1 millimeters, 50±1 millimeters, 60±1 millimeters, 70±1 millimeters, 80±1 millimeters. In many configurations, a shunt of the disclosure has an undistorted, unstretched, length (105) of about 2 centimeters (cm) to about 7 cm, from about 2.5 cm to about 7 cm, from about 3 cm to about 7 cm, from about 3.5 cm to about 7 cm, from about 4 cm to about 7 cm, from about 4.5 cm to about 7 cm, or from about 5 cm to about 7 cm. In many configurations, a shunt of the disclosure has an undistorted, unstretched, length (105) that is no longer than about 8 cm, no longer than about 7 cm, no longer than about 6 cm, no longer than about 5 cm, or no longer than about 4 cm. In specific configurations, the hollow tube has a length of about 4 cm.

In some configurations, a hollow tube of the disclosure has an extended length of 30±1 millimeter, 31±1 millimeter, 32±1 millimeter, 33±1 millimeter, 34±1 millimeter, 35±1 millimeter, 36±1 millimeter, 37±1 millimeter, 38±1 millimeter, 39±1 millimeter, 40±1 millimeter, 41±1 millimeter, 42±1 millimeter, 43±1 millimeter, 44±1 millimeter, 45±1 millimeter, 46±1 millimeter, 47±1 millimeter, 48±1 millimeter, 49±1 millimeter, 50±1 millimeter, 51±1 millimeter, 52±1 millimeter, 53±1 millimeter, 54±1 millimeter, 55±1 millimeter, 56±1 millimeter, 57±1 millimeter, 58±1 millimeter, 59±1 millimeter, 60±1 millimeter, 61±1 millimeter, 62±1 millimeter, 63±1 millimeter, 64±1 millimeter, 65=1 millimeter, 66±1 millimeter, 67±1 millimeter, 68±1 millimeter, 69±1 millimeter, 70±1 millimeter, 71±1 millimeter, 72±1 millimeter, 73±1 millimeter, 74±1 millimeter, 75±1 millimeter, 76±1 millimeter, 77±1 millimeter, 78±1 millimeter, 79±1 millimeter, 80±1 millimeter, 81±1 millimeter, 82±1 millimeter, 83±1 millimeter, 84±1 millimeter, 85±1 millimeter, 86±1 millimeter, 87±1 millimeter, 88±1 millimeter, 89±1 millimeter, 90±1 millimeter, 91±1 millimeter, 92±1 millimeter, 93±1 millimeter, 94±1 millimeter, 95±1 millimeter, 96±1 millimeter, 97±1 millimeter, 98±1 millimeter, 99±1 millimeter, 100±1 millimeter, 101±1 millimeter, 102±1 millimeter, 103±1 millimeter, 104±1 millimeter, 105±1 millimeter, 106±1 millimeter, 107±1 millimeter, 108±1 millimeter, 109±1 millimeter, 110±1 millimeter, 111±1 millimeter, 112±1 millimeter, 113±1 millimeter, 114±1 millimeter, 115±1 millimeter, 116±1 millimeter, 117±1 millimeter, 118±1 millimeter, 119±1 millimeter, 120±1 millimeter, or another suitable length.

In some configurations, a shunt of the disclosure comprises a plurality of side perforation(s) at the proximal end (barb end) of around 3-5 Fr. In some embodiments, the present device does not comprise any perforation in the intermediary segment. Without being bound by theory, it is contemplated that sizing the tube at 3 Fr size may facilitate deployment through a smaller port(s) and may reduce the risk of pregnancy complications such as leakage of fluid. The plurality of perforations can have any pattern, e.g., spiral, serial, sequential, at defined angles, random, or another suitable patterns. In many configurations, one or a plurality of perforations are present in the proximal end of the stent of a stent of the disclosure in any pattern described above. The perforations can be placed at intervals of about every 1 millimeter, every 2 millimeters, every 3 millimeters, every 4 millimeters, and/or every 5 millimeters from one another along the length of the proximal pigtail. In some configurations of the shunt, the plurality of perforations are present on the shunt at every about 60 degrees, 61 degrees, 62 degrees, 63 degrees, 64 degrees, 65 degrees, 66 degrees, 67 degrees, 68 degrees, 69 degrees, 70 degrees, 71 degrees, 72 degrees, 73 degrees, 74 degrees, 75 degrees, 76 degrees, 77 degrees, 78 degrees, 79 degrees, 80 degrees, 81 degrees, 82 degrees, 83 degrees, 84 degrees, 85 degrees, 86 degrees, 87 degrees, 88 degrees, 89 degrees, 90 degrees, 91 degrees, 92 degrees, 93 degrees, 94 degrees, 95 degrees, 96 degrees, 97 degrees, 98 degrees, 99 degrees, 100 degrees, 101 degrees, 102 degrees, 103 degrees, 104 degrees, 105 degrees, 106 degrees, 107 degrees, 108 degrees, 109 degrees, 110 degrees, 111 degrees, 112 degrees, 113 degrees, 114 degrees, 115 degrees, 116 degrees, 117 degrees, 118 degrees, 119 degrees, 120 degrees, 121 degrees, 122 degrees, 123 degrees, 124 degrees, 125 degrees, 126 degrees, 127 degrees, 128 degrees, 129 degrees, 130 degrees, 131 degrees, 132 degrees, 133 degrees, 134 degrees, 135 degrees, 136 degrees, 137 degrees, 138 degrees, 139 degrees, 140 degrees, 141 degrees, 142 degrees, 143 degrees, 144 degrees, 145 degrees, 146 degrees, 147 degrees, 148 degrees, 149 degrees, 150 degrees, 151 degrees, 152 degrees, 153 degrees, 154 degrees, 155 degrees, 156 degrees, 157 degrees, 158 degrees, 159 degrees, 160 degrees, 161 degrees, 162 degrees, 163 degrees, 164 degrees, 165 degrees, 166 degrees, 167 degrees, 168 degrees, 169 degrees, 170 degrees, 171 degrees, 172 degrees, 173 degrees, 174 degrees, 175 degrees, 176 degrees, 177 degrees, 178 degrees, 179 degrees, 180 degrees, 181 degrees, 182 degrees, 183 degrees, 184 degrees, 185 degrees, 186 degrees, 187 degrees, 189 degrees, and/or at about every 190 degrees from one another.

In some configurations, a shunt of the disclosure comprises at least 3 perforations within the proximal pigtail, at least 4 perforations, at least 5 perforations, at least 6 perforations, at least 7 perforations, at least 9 perforations, at least 10 perforations, at least 11 perforations, at least 12 perforations, at least 13 perforations, at least 14 perforations, at least 15 perforations, at least 16 perforations, at least 17 perforations, at least 18 perforations, at least 19 perforations, at least 20 perforations, at least 21 perforations, at least 22 perforations, at least 23 perforations, at least 24 perforations, at least 25 perforations, at least 26 perforations, at least 27 perforations, at least 28 perforations, at least 29 perforations, at least 30 perforations, at least 31 perforations, at least 32 perforations, at least 33 perforations, at least 34 perforations, at least 35 perforations, at least 36 perforations, at least 37 perforations, at least 38 perforations, at least 39 perforations, at least 40 perforations, at least 41 perforations, at least 42 perforations, at least 43 perforations, at least 44 perforations, at least 45 perforations, at least 46 perforations, at least 47 perforations, at least 48 perforations, at least 49 perforations, at least 50 perforations, or another suitable number. In some embodiments, the present device does not comprise any perforation in the intermediary segment.

In some configurations, a shunt of the disclosure comprises no more than 3 perforations, no more than 4 perforations, no more than 5 perforations, no more than 6 perforations, no more than 7 perforations, no more than 8 perforations, no more than 9 perforations, no more than 10 perforations, no more than 11 perforations, no more than 12 perforations, no more than 13 perforations, no more than 14 perforations, no more than 15 perforations, no more than 16 perforations, no more than 17 perforations, no more than 18 perforations, no more than 19 perforations, no more than 20 perforations, no more than 21 perforations, no more than 22 perforations, no more than 23 perforations, no more than 24 perforations, no more than 25 perforations, no more than 26 perforations, no more than 27 perforations, no more than 28 perforations, no more than 29 perforations, no more than 30 perforations, no more than 31 perforations, no more than 32 perforations, no more than 33 perforations, no more than 34 perforations, no more than 35 perforations, no more than 36 perforations, no more than 37 perforations, no more than 38 perforations, no more than 39 perforations, no more than 40 perforations, no more than 41 perforations, no more than 42 perforations, no more than 43 perforations, no more than 44 perforations, no more than 45 perforations, no more than 46 perforations, no more than 47 perforations, no more than 48 perforations, no more than 49 perforations, no more than 50 perforations, or another suitable number. In some embodiments, the present device does not comprise any perforation in the intermediary segment.

It is contemplated that the shunts of the disclosure have a firm but flexible barb at one end, generally the end referred to as the proximal end. It is also contemplated that the shunts of the disclosure comprise a softer pigtail at the proximal end, the distal end, or both. It is contemplated that a stiffness of the mid segment is optimal for maintaining its form during use, i.e., not becoming obstructed, kinked, or otherwise impeded. In some configurations, shunts of the disclosure are characterized by a softer material at the pigtail comprising a plurality of perforations. The softer material may be designed to better place the stent to accommodate within the bladder or chest along the tissue contours and to decrease a risk of perforation of bladder wall or lung tissue.

It is contemplated that the softer material is softer with respect to a rigidity of the intermediary segment or the hollow tube. For instance, a tensile strength of the softer material may be less than a tensile strength of the intermediary segment or the hollow tube. In some embodiments, the tensile strength of the softer material (i.e., the tensile strength of the softer pigtail at the proximal end, the distal end, or both) may be at least about 5 megapascals (MPa), 6 MPa, 7 MPa, 8 MPa, 9 MPa, 10 MPa, 11 MPa, 12 MPa, 13 MPa, 14 MPa, 15 MPa, 16 MPa, 17 MPa, 18 MPa, 19 MPa, 20 MPa, or more, at most about 20 MPa, 19 MPa, 18 MPa, 17 MPa, 16 MPa, 15 MPa, 14 MPa, 13 MPa, 12 MPa, 11 MPa, 10 MPa, 9 MPa, 8 MPa, 7 MPa, 6 MPa, 5 MPa, or less, or within a range defined by any two of the preceding values. For instance, the tensile strength of the softer material may be between about 5 MPa and about 6 MPa, about 5 MPa and about 7 MPa, about 5 MPa and about 8 MPa, about 5 MPa and about 9 MPa, about 5 MPa and about 10 MPa, about 5 MPa and about 11 MPa, about 5 MPa and about 12 MPa, about 5 MPa and about 13 MPa, about 5 MPa and about 14 MPa, about 5 MPa and about 15 MPa, about 5 MPa and about 16 MPa, about 5 MPa and about 17 MPa, about 5 MPa and about 18 MPa, about 5 MPa and about 19 MPa, about 5 MPa and about 20 MPa, about 6 MPa and about 7 MPa, about 6 MPa and about 8 MPa, about 6 MPa and about 9 MPa, about 6 MPa and about 10 MPa, about 6 MPa and about 11 MPa, about 6 MPa and about 12 MPa, about 6 MPa and about 13 MPa, about 6 MPa and about 14 MPa, about 6 MPa and about 15 MPa, about 6 MPa and about 16 MPa, about 6 MPa and about 17 MPa, about 6 MPa and about 18 MPa, about 6 MPa and about 19 MPa, about 6 MPa and about 20 MPa, about 7 MPa and about 8 MPa, about 7 MPa and about 9 MPa, about 7 MPa and about 10 MPa, about 7 MPa and about 11 MPa, about 7 MPa and about 12 MPa, about 7 MPa and about 13 MPa, about 7 MPa and about 14 MPa, about 7 MPa and about 15 MPa, about 7 MPa and about 16 MPa, about 7 MPa and about 17 MPa, about 7 MPa and about 18 MPa, about 7 MPa and about 19 MPa, about 7 MPa and about 20 MPa, about 8 MPa and about 9 MPa, about 8 MPa and about 10 MPa, about 8 MPa and about 11 MPa, about 8 MPa and about 12 MPa, about 8 MPa and about 13 MPa, about 8 MPa and about 14 MPa, about 8 MPa and about 15 MPa, about 8 MPa and about 16 MPa, about 8 MPa and about 17 MPa, about 8 MPa and about 18 MPa, about 8 MPa and about 19 MPa, about 8 MPa and about 20 MPa, about 9 MPa and about 10 MPa, about 9 MPa and about 11 MPa, about 9 MPa and about 12 MPa, about 9 MPa and about 13 MPa, about 9 MPa and about 14 MPa, about 9 MPa and about 15 MPa, about 9 MPa and about 16 MPa, about 9 MPa and about 17 MPa, about 9 MPa and about 18 MPa, about 9 MPa and about 19 MPa, about 9 MPa and about 20 MPa, about 10 MPa and about 11 MPa, about 10 MPa and about 12 MPa, about 10 MPa and about 13 MPa, about 10 MPa and about 14 MPa, about 10 MPa and about 15 MPa, about 10 MPa and about 16 MPa, about 10 MPa and about 17 MPa, about 10 MPa and about 18 MPa, about 10 MPa and about 19 MPa, about 10 MPa and about 20 MPa, about 11 MPa and about 12 MPa, about 11 MPa and about 13 MPa, about 11 MPa and about 14 MPa, about 11 MPa and about 15 MPa, about 11 MPa and about 16 MPa, about 11 MPa and about 17 MPa, about 11 MPa and about 18 MPa, about 11 MPa and about 19 MPa, about 11 MPa and about 20 MPa, about 12 MPa and about 13 MPa, about 12 MPa and about 14 MPa, about 12 MPa and about 15 MPa, about 12 MPa and about 16 MPa, about 12 MPa and about 17 MPa, about 12 MPa and about 18 MPa, about 12 MPa and about 19 MPa, about 12 MPa and about 20 MPa, about 13 MPa and about 14 MPa, about 13 MPa and about 15 MPa, about 13 MPa and about 16 MPa, about 13 MPa and about 17 MPa, about 13 MPa and about 18 MPa, about 13 MPa and about 19 MPa, about 13 MPa and about 20 MPa, about 14 MPa and about 15 MPa, about 14 MPa and about 16 MPa, about 14 MPa and about 17 MPa, about 14 MPa and about 18 MPa, about 14 MPa and about 19 MPa, about 14 MPa and about 20 MPa, about 15 MPa and about 16 MPa, about 15 MPa and about 17 MPa, about 15 MPa and about 18 MPa, about 15 MPa and about 19 MPa, about 15 MPa and about 20 MPa, about 16 MPa and about 17 MPa, about 16 MPa and about 18 MPa, about 16 MPa and about 19 MPa, about 16 MPa and about 20 MPa, about 17 MPa and about 18 MPa, about 17 MPa and about 19 MPa, about 17 MPa and about 20 MPa, about 18 MPa and about 19 MPa, about 18 MPa and about 20 MPa, or about 19 MPa and about 20 MPa.

By contrast, the tensile strength of the intermediary segment or the hollow tube may be at least about 25 MPa, 26 MPa, 27 MPa, 28 MPa, 29 MPa, 30 MPa, 31 MPa, 32 MPa, 33 MPa, 34 MPa, 35 MPa, 36 MPa, 37 MPa, 38 MPa, 39 MPa, 40 MPa, 41 MPa, 42 MPa, 43 MPa, 44 MPa, 45 MPa, 46 MPa, 47 MPa, 48 MPa, 49 MPa, 50 MPa, or more, at most about 50 MPa, 49 MPa, 48 MPa, 47 MPa, 46 MPa, 45 MPa, 44 MPa, 43 MPa, 42 MPa, 41 MPa, 40 MPa, 39 MPa, 38 MPa, 37 MPa, 36 MPa, 35 MPa, 34 MPa, 33 MPa, 32 MPa, 31 MPa, 30 MPa, 29 MPa, 28 MPa, 27 MPa, 26 MPa, 25 MPa, or less, or within a range defined by any two of the preceding values. For instance, the tensile strength of the intermediary segment or the hollow tube may be between about 25 MPa and about 26 MPa, about 25 MPa and about 27 MPa, about 25 MPa and about 28 MPa, about 25 MPa and about 29 MPa, about 25 MPa and about 30 MPa, about 25 MPa and about 31 MPa, about 25 MPa and about 32 MPa, about 25 MPa and about 33 MPa, about 25 MPa and about 34 MPa, about 25 MPa and about 35 MPa, about 25 MPa and about 36 MPa, about 25 MPa and about 37 MPa, about 25 MPa and about 38 MPa, about 25 MPa and about 39 MPa, about 25 MPa and about 40 MPa, about 25 MPa and about 41 MPa, about 25 MPa and about 42 MPa, about 25 MPa and about 43 MPa, about 25 MPa and about 44 MPa, about 25 MPa and about 45 MPa, about 25 MPa and about 46 MPa, about 25 MPa and about 47 MPa, about 25 MPa and about 48 MPa, about 25 MPa and about 49 MPa, about 25 MPa and about 50 MPa, about 26 MPa and about 27 MPa, about 26 MPa and about 28 MPa, about 26 MPa and about 29 MPa, about 26 MPa and about 30 MPa, about 26 MPa and about 31 MPa, about 26 MPa and about 32 MPa, about 26 MPa and about 33 MPa, about 26 MPa and about 34 MPa, about 26 MPa and about 35 MPa, about 26 MPa and about 36 MPa, about 26 MPa and about 37 MPa, about 26 MPa and about 38 MPa, about 26 MPa and about 39 MPa, about 26 MPa and about 40 MPa, about 26 MPa and about 41 MPa, about 26 MPa and about 42 MPa, about 26 MPa and about 43 MPa, about 26 MPa and about 44 MPa, about 26 MPa and about 45 MPa, about 26 MPa and about 46 MPa, about 26 MPa and about 47 MPa, about 26 MPa and about 48 MPa, about 26 MPa and about 49 MPa, about 26 MPa and about 50 MPa, about 27 MPa and about 28 MPa, about 27 MPa and about 29 MPa, about 27 MPa and about 30 MPa, about 27 MPa and about 31 MPa, about 27 MPa and about 32 MPa, about 27 MPa and about 33 MPa, about 27 MPa and about 34 MPa, about 27 MPa and about 35 MPa, about 27 MPa and about 36 MPa, about 27 MPa and about 37 MPa, about 27 MPa and about 38 MPa, about 27 MPa and about 39 MPa, about 27 MPa and about 40 MPa, about 27 MPa and about 41 MPa, about 27 MPa and about 42 MPa, about 27 MPa and about 43 MPa, about 27 MPa and about 44 MPa, about 27 MPa and about 45 MPa, about 27 MPa and about 46 MPa, about 27 MPa and about 47 MPa, about 27 MPa and about 48 MPa, about 27 MPa and about 49 MPa, about 27 MPa and about 50 MPa, about 28 MPa and about 29 MPa, about 28 MPa and about 30 MPa, about 28 MPa and about 31 MPa, about 28 MPa and about 32 MPa, about 28 MPa and about 33 MPa, about 28 MPa and about 34 MPa, about 28 MPa and about 35 MPa, about 28 MPa and about 36 MPa, about 28 MPa and about 37 MPa, about 28 MPa and about 38 MPa, about 28 MPa and about 39 MPa, about 28 MPa and about 40 MPa, about 28 MPa and about 41 MPa, about 28 MPa and about 42 MPa, about 28 MPa and about 43 MPa, about 28 MPa and about 44 MPa, about 28 MPa and about 45 MPa, about 28 MPa and about 46 MPa, about 28 MPa and about 47 MPa, about 28 MPa and about 48 MPa, about 28 MPa and about 49 MPa, about 28 MPa and about 50 MPa, about 29 MPa and about 30 MPa, about 29 MPa and about 31 MPa, about 29 MPa and about 32 MPa, about 29 MPa and about 33 MPa, about 29 MPa and about 34 MPa, about 29 MPa and about 35 MPa, about 29 MPa and about 36 MPa, about 29 MPa and about 37 MPa, about 29 MPa and about 38 MPa, about 29 MPa and about 39 MPa, about 29 MPa and about 40 MPa, about 29 MPa and about 41 MPa, about 29 MPa and about 42 MPa, about 29 MPa and about 43 MPa, about 29 MPa and about 44 MPa, about 29 MPa and about 45 MPa, about 29 MPa and about 46 MPa, about 29 MPa and about 47 MPa, about 29 MPa and about 48 MPa, about 29 MPa and about 49 MPa, about 29 MPa and about 50 MPa, about 30 MPa and about 31 MPa, about 30 MPa and about 32 MPa, about 30 MPa and about 33 MPa, about 30 MPa and about 34 MPa, about 30 MPa and about 35 MPa, about 30 MPa and about 36 MPa, about 30 MPa and about 37 MPa, about 30 MPa and about 38 MPa, about 30 MPa and about 39 MPa, about 30 MPa and about 40 MPa, about 30 MPa and about 41 MPa, about 30 MPa and about 42 MPa, about 30 MPa and about 43 MPa, about 30 MPa and about 44 MPa, about 30 MPa and about 45 MPa, about 30 MPa and about 46 MPa, about 30 MPa and about 47 MPa, about 30 MPa and about 48 MPa, about 30 MPa and about 49 MPa, about 30 MPa and about 50 MPa, about 31 MPa and about 32 MPa, about 31 MPa and about 33 MPa, about 31 MPa and about 34 MPa, about 31 MPa and about 35 MPa, about 31 MPa and about 36 MPa, about 31 MPa and about 37 MPa, about 31 MPa and about 38 MPa, about 31 MPa and about 39 MPa, about 31 MPa and about 40 MPa, about 31 MPa and about 41 MPa, about 31 MPa and about 42 MPa, about 31 MPa and about 43 MPa, about 31 MPa and about 44 MPa, about 31 MPa and about 45 MPa, about 31 MPa and about 46 MPa, about 31 MPa and about 47 MPa, about 31 MPa and about 48 MPa, about 31 MPa and about 49 MPa, about 31 MPa and about 50 MPa, about 32 MPa and about 33 MPa, about 32 MPa and about 34 MPa, about 32 MPa and about 35 MPa, about 32 MPa and about 36 MPa, about 32 MPa and about 37 MPa, about 32 MPa and about 38 MPa, about 32 MPa and about 39 MPa, about 32 MPa and about 40 MPa, about 32 MPa and about 41 MPa, about 32 MPa and about 42 MPa, about 32 MPa and about 43 MPa, about 32 MPa and about 44 MPa, about 32 MPa and about 45 MPa, about 32 MPa and about 46 MPa, about 32 MPa and about 47 MPa, about 32 MPa and about 48 MPa, about 32 MPa and about 49 MPa, about 32 MPa and about 50 MPa, about 33 MPa and about 34 MPa, about 33 MPa and about 35 MPa, about 33 MPa and about 36 MPa, about 33 MPa and about 37 MPa, about 33 MPa and about 38 MPa, about 33 MPa and about 39 MPa, about 33 MPa and about 40 MPa, about 33 MPa and about 41 MPa, about 33 MPa and about 42 MPa, about 33 MPa and about 43 MPa, about 33 MPa and about 44 MPa, about 33 MPa and about 45 MPa, about 33 MPa and about 46 MPa, about 33 MPa and about 47 MPa, about 33 MPa and about 48 MPa, about 33 MPa and about 49 MPa, about 33 MPa and about 50 MPa, about 34 MPa and about 35 MPa, about 34 MPa and about 36 MPa, about 34 MPa and about 37 MPa, about 34 MPa and about 38 MPa, about 34 MPa and about 39 MPa, about 34 MPa and about 40 MPa, about 34 MPa and about 41 MPa, about 34 MPa and about 42 MPa, about 34 MPa and about 43 MPa, about 34 MPa and about 44 MPa, about 34 MPa and about 45 MPa, about 34 MPa and about 46 MPa, about 34 MPa and about 47 MPa, about 34 MPa and about 48 MPa, about 34 MPa and about 49 MPa, about 34 MPa and about 50 MPa, about 35 MPa and about 36 MPa, about 35 MPa and about 37 MPa, about 35 MPa and about 38 MPa, about 35 MPa and about 39 MPa, about 35 MPa and about 40 MPa, about 35 MPa and about 41 MPa, about 35 MPa and about 42 MPa, about 35 MPa and about 43 MPa, about 35 MPa and about 44 MPa, about 35 MPa and about 45 MPa, about 35 MPa and about 46 MPa, about 35 MPa and about 47 MPa, about 35 MPa and about 48 MPa, about 35 MPa and about 49 MPa, about 35 MPa and about 50 MPa, about 36 MPa and about 37 MPa, about 36 MPa and about 38 MPa, about 36 MPa and about 39 MPa, about 36 MPa and about 40 MPa, about 36 MPa and about 41 MPa, about 36 MPa and about 42 MPa, about 36 MPa and about 43 MPa, about 36 MPa and about 44 MPa, about 36 MPa and about 45 MPa, about 36 MPa and about 46 MPa, about 36 MPa and about 47 MPa, about 36 MPa and about 48 MPa, about 36 MPa and about 49 MPa, about 36 MPa and about 50 MPa, about 37 MPa and about 38 MPa, about 37 MPa and about 39 MPa, about 37 MPa and about 40 MPa, about 37 MPa and about 41 MPa, about 37 MPa and about 42 MPa, about 37 MPa and about 43 MPa, about 37 MPa and about 44 MPa, about 37 MPa and about 45 MPa, about 37 MPa and about 46 MPa, about 37 MPa and about 47 MPa, about 37 MPa and about 48 MPa, about 37 MPa and about 49 MPa, about 37 MPa and about 50 MPa, about 38 MPa and about 39 MPa, about 38 MPa and about 40 MPa, about 38 MPa and about 41 MPa, about 38 MPa and about 42 MPa, about 38 MPa and about 43 MPa, about 38 MPa and about 44 MPa, about 38 MPa and about 45 MPa, about 38 MPa and about 46 MPa, about 38 MPa and about 47 MPa, about 38 MPa and about 48 MPa, about 38 MPa and about 49 MPa, about 38 MPa and about 50 MPa, about 39 MPa and about 40 MPa, about 39 MPa and about 41 MPa, about 39 MPa and about 42 MPa, about 39 MPa and about 43 MPa, about 39 MPa and about 44 MPa, about 39 MPa and about 45 MPa, about 39 MPa and about 46 MPa, about 39 MPa and about 47 MPa, about 39 MPa and about 48 MPa, about 39 MPa and about 49 MPa, about 39 MPa and about 50 MPa, about 40 MPa and about 41 MPa, about 40 MPa and about 42 MPa, about 40 MPa and about 43 MPa, about 40 MPa and about 44 MPa, about 40 MPa and about 45 MPa, about 40 MPa and about 46 MPa, about 40 MPa and about 47 MPa, about 40 MPa and about 48 MPa, about 40 MPa and about 49 MPa, about 40 MPa and about 50 MPa, about 41 MPa and about 42 MPa, about 41 MPa and about 43 MPa, about 41 MPa and about 44 MPa, about 41 MPa and about 45 MPa, about 41 MPa and about 46 MPa, about 41 MPa and about 47 MPa, about 41 MPa and about 48 MPa, about 41 MPa and about 49 MPa, about 41 MPa and about 50 MPa, about 42 MPa and about 43 MPa, about 42 MPa and about 44 MPa, about 42 MPa and about 45 MPa, about 42 MPa and about 46 MPa, about 42 MPa and about 47 MPa, about 42 MPa and about 48 MPa, about 42 MPa and about 49 MPa, about 42 MPa and about 50 MPa, about 43 MPa and about 44 MPa, about 43 MPa and about 45 MPa, about 43 MPa and about 46 MPa, about 43 MPa and about 47 MPa, about 43 MPa and about 48 MPa, about 43 MPa and about 49 MPa, about 43 MPa and about 50 MPa, about 44 MPa and about 45 MPa, about 44 MPa and about 46 MPa, about 44 MPa and about 47 MPa, about 44 MPa and about 48 MPa, about 44 MPa and about 49 MPa, about 44 MPa and about 50 MPa, about 45 MPa and about 46 MPa, about 45 MPa and about 47 MPa, about 45 MPa and about 48 MPa, about 45 MPa and about 49 MPa, about 45 MPa and about 50 MPa, about 46 MPa and about 47 MPa, about 46 MPa and about 48 MPa, about 46 MPa and about 49 MPa, about 46 MPa and about 50 MPa, about 47 MPa and about 48 MPa, about 47 MPa and about 49 MPa, about 47 MPa and about 50 MPa, about 48 MPa and about 49 MPa, about 48 MPa and about 50 MPa, or about 49 MPa and about 50 MPa.

In some embodiments, use of a softer material for the proximal end may reduce the risk of perforation or erosion within sensitive organs such as the bladder, bowel, lungs, heart, diaphragm, or liver.

In some embodiments, a firmer material may be used for the distal end when the shunt is placed in the lateral or posterior chest. In such cases, the firmer material may help the shunt to stay close to the chest wall and minimize or reduce dislocation of the shunt into the chest cavity.

In some embodiments, a softer material may be used for the distal end when the shunt is placed in the bladder or anterior chest. In such cases, the softer material may yield to movement (such as hand or finger movement from the fetus), reducing the risk of entanglement and allowing freeing of the shunt should it become caught.

It is contemplated that the shunts of the disclosure may weigh between 0.004 grams (g) to 3 g, 0.005 g to 3 g, 0.006 g to 3 g, 0.007 g to 3 g, 0.008 g to 3 g, 0.009 g to 3 g, 0.01 g to 3 g, 0.02 g to 3 g, 0.03 g to 3 g, 0.04 g to 3 g, 0.05 g to 3 g, 0.06 g to 3 g, 0.07 g to 3 g, 0.08 g to 3 g, 0.09 g to 3 g, 0.11 g to 3 g, 0.12 g to 3 g, 0.13 g to 3 g, 0.14 g to 3 g, 0.15 g to 3 g, 0.16 g to 3 g, 0.17 g to 3 g, 0.18 g to 3 g, 0.19 g to 3 g, 0.2 g to 3 g, 0.21 g to 3 g, 0.22 g to 3 g, 0.23 g to 3 g, 0.24 g to 3 g, 0.25 g to 3 g, 0.26 g to 3 g, 0.27 g to 3 g, 0.28 g to 3 g, 0.29 g to 3 g, 0.3 g to 3 g, 0.31 g to 3 g, 0.32 g to 3 g, 0.33 g to 3 g, 0.34 g to 3 g, 0.35 g to 3 g, 0.36 g to 3 g, 0.37 g to 3 g, 0.38 g to 3 g, 0.39 g to 3 g, 0.40 g to 3 g, 0.41 g to 3 g, 0.42 g to 3 g, 0.43 g to 3 g, 0.44 g to 3 g, 0.45 g to 3 g, 0.46 g to 3 g, 0.47 g to 3 g, 0.48 g to 3 g, 0.49 g to 3 g, 0.5 g to 3 g, 0.004 g to 2 g, 0.005 g to 2 g, 0.006 g to 2 g, 0.007 g to 2 g, 0.008 g to 2 g, 0.009 g to 2 g, 0.01 g to 2 g, 0.02 g to 2 g, 0.03 g to 2 g, 0.04 g to 2 g, 0.05 g to 2 g, 0.06 g to 2 g, 0.07 g to 2 g, 0.08 g to 2 g, 0.09 g to 2 g, 0.11 g to 2 g, 0.12 g to 2 g, 0.13 g to 2 g, 0.14 g to 2 g, 0.15 g to 2 g, 0.16 g to 2 g, 0.17 g to 2 g, 0.18 g to 2 g, 0.19 g to 2 g, 0.2 g to 2 g, 0.21 g to 2 g, 0.22 g to 2 g, 0.23 g to 2 g, 0.24 g to 2 g, 0.25 g to 2 g, 0.26 g to 2 g, 0.27 g to 2 g, 0.28 g to 2 g, 0.29 g to 2 g, 0.3 g to 2 g, 0.31 g to 2 g, 0.32 g to 2 g, 0.33 g to 2 g, 0.34 g to 2 g, 0.35 g to 2 g, 0.36 g to 2 g, 0.37 g to 2 g, 0.38 g to 2 g, 0.39 g to 2 g, 0.40 g to 2 g, 0.41 g to 2 g, 0.42 g to 2 g, 0.43 g to 2 g, 0.44 g to 2 g, 0.45 g to 2 g, 0.46 g to 2 g, 0.47 g to 2 g, 0.48 g to 2 g, 0.49 g to 2 g, or 0.5 g to 2 g. In some particular configurations, the device weighs about 0.15 g. Preferably, it is contemplated that a shunt of the disclosure weighs within a range of 0.05 g to 2 g.

The present disclosure provides a stent/shunt comprising at least one barbed end placed between the intermediary section and the proximal end. In some configurations, the shunts described herein can comprises two or more, three or more, four or more, five or more, six or more, seven or more, and/or eight or more barbed ends. In some configurations, the shunts described herein can comprise no more than one, no more than two, no more than three, no more than four, no more than five, no more than six, no more than seven, and/or no more than eight barbed ends. In some instances, such barb ends can be placed, e.g., sequentially between the intermediary section and the proximal end. Alternatively, such barbed ends can be placed, e.g., sequentially between the intermediary section and the distal end. Further, such barbed ends can be placed, e.g., in any order between the intermediary section and either the distal end or the proximal end. It is contemplated that in most configurations barded end(s) are not placed in the intermediary segment. In some configurations of the shunt, barbed ends can be placed within about 60 degrees, 61 degrees, 62 degrees, 63 degrees, 64 degrees, 65 degrees, 66 degrees, 67 degrees, 68 degrees, 69 degrees, 70 degrees, 71 degrees, 72 degrees, 73 degrees, 74 degrees, 75 degrees, 76 degrees, 77 degrees, 78 degrees, 79 degrees, 80 degrees, 81 degrees, 82 degrees, 83 degrees, 84 degrees, 85 degrees, 86 degrees, 87 degrees, 88 degrees, 89 degrees, 90 degrees, 91 degrees, 92 degrees, 93 degrees, 94 degrees, 95 degrees, 96 degrees, 97 degrees, 98 degrees, 99 degrees, 100 degrees, 101 degrees, 102 degrees, 103 degrees, 104 degrees, 105 degrees, 106 degrees, 107 degrees, 108 degrees, 109 degrees, 110 degrees, 111 degrees, 112 degrees, 113 degrees, 114 degrees, 115 degrees, 116 degrees, 117 degrees, 118 degrees, 119 degrees, 120 degrees, 121 degrees, 122 degrees, 123 degrees, 124 degrees, 125 degrees, 126 degrees, 127 degrees, 128 degrees, 129 degrees, 130 degrees, 131 degrees, 132 degrees, 133 degrees, 134 degrees, 135 degrees, 136 degrees, 137 degrees, 138 degrees, 139 degrees, 140 degrees, 141 degrees, 142 degrees, 143 degrees, 144 degrees, 145 degrees, 146 degrees, 147 degrees, 148 degrees, 149 degrees, 150 degrees, 151 degrees, 152 degrees, 153 degrees, 154 degrees, 155 degrees, 156 degrees, 157 degrees, 158 degrees, 159 degrees, 160 degrees, 161 degrees, 162 degrees, 163 degrees, 164 degrees, 165 degrees, 166 degrees, 167 degrees, 168 degrees, 169 degrees, 170 degrees, 171 degrees, 172 degrees, 173 degrees, 174 degrees, 175 degrees, 176 degrees, 177 degrees, 178 degrees, 179 degrees, 180 degrees, 181 degrees, 182 degrees, 183 degrees, 184 degrees, 185 degrees, 186 degrees, 187 degrees, 189 degrees, and/or within 190 degrees from one another. In many configurations, barbed ends can be placed within about 1 millimeter, 2 millimeters, 3 millimeters, 4 millimeters, and or 5 millimeters of one another. It is contemplated that an optional configuration of the device having one barb end at the proximal end and another barbed end at the opposite site (i.e., distal end), e.g., approximately 3-4 mm away may function as a second checkpoint in the event of manipulation occurring due to fetal movement.

In some aspects, a shunt of the disclosure is sized by gauge measured by French gauge (Fr) size. For example, a “3 French” gauge of the disclosure is characterized by an outer diameter of the hollow tube of approximately 1 mm and a circumference of approximately 3.14 mm; a “4 French” gauge of the disclosure is characterized by an outer diameter of the hollow tube of approximately 1.33 mm and a circumference of approximately 4.19 mm; a “5 French” gauge of the disclosure is characterized by an outer diameter of the hollow tube of approximately 1.67 mm and a circumference of approximately 3.14 mm; a “4 French” gauge of the disclosure is characterized by an outer diameter of the hollow tube of approximately 1.33 mm and a circumference of approximately 5.24 mm. In some configurations, a shunt of the disclosure has a plurality of side perforations at the proximal end (barb end) of a, e.g., the 5 Fr 4 cm device (5Fr 4 cm device means a device has an outer diameter of 5 French and a length of 4 cm. In some configurations, a shunt of the disclosure has can be a 3 Fr device that is between 2 cm and 4 cm in length, with one barb at one end and a pigtail on the other end, having no perforation in the intermediary segment. In some configurations, a shunt of the disclosure comprises one or more optional perforations in the intermediary segment.

A shunt of the disclosure is generally manufactured from a polymer. Non-limiting examples of polymers contemplated by the disclosure for manufacture of the device include polymers that can be manipulated for formation of the desired pigcoils, without undergoing any significant alteration to its form after implantation. Examples of polymers contemplated by the disclosure include polyethylene (PE), including High-Density Polyethylene (HDPE) and Low-Density Polyethylene (LDPE); polypropylene (PP); polyvinyl chloride (PVC); polystyrene (PS); polyethylene terephthalate (PET); polyamide (Nylon); polycarbonate (PC); acrylonitrile butadiene styrene (ABS); poly(methyl methacrylate) (PMMA); or polyvinylidene fluoride (PVDF): PVDF.

### Manufacture of a Stent of the Disclosure

3D printing, also known as additive manufacturing, can be used for manufacturing a shunt of the disclosure, particularly a shunt that is fabricated with specifications tailored to individual subjects' needs (e.g., specifications tailored to the unique fetal dimension in need of shunting). 3D printing or additive manufacturing is the construction of a 3D object from a computer aided design (CAD) model or a digital 3D model. It can be done in a variety of processes in which material is deposited, joined or solidified under computer control, with the material being added together (such as plastics, liquids or powder grains being fused), typically layer by layer. Fused deposition modeling (FDM), which uses a continuous filament of a thermoplastic material, can be used to manufacture a shunt of the disclosure.

The present disclosure contemplates devices that are manufactured with 3D printing. The steps of the 3D printing manufacture are as follows:

Design: a digital 3D model, created using CAD software or generated from medical imaging data like magnetic resonance imaging (MRI), computed tomography (CT), or positron emission tomography (PET) scans of a subject is used to determine the dimensions of the device and the specifications (see e.g., Example 1).

Printing: The 3D printer builds the device layer by layer from the bottom up. It adds material where needed according to the 3D model. The 3D printer can print with different thicknesses or different materials, thereby supporting manufacturing of a device that is “softer” or “harder” in some areas.

Post-Processing: After printing, the device may undergo cleaning, curing, or sterilization, depending on the material and intended use. For a fetal shunt, washing and sterilization can be considered prior to use.

In some aspects, the disclosure describes a stent/shunt and methods for using the same in shunting, particularly fetal shunting. Fetal shunting is a specialized medical procedure used to treat certain conditions in fetuses, particularly those involving fluid accumulation in areas such as the chest or abdomen. The disclosure contemplates methods of shunting where the condition requiring fetal shunting is diagnosed through prenatal imaging techniques like ultrasound. A detailed plan is made by the medical team, considering the fetus's condition, the location of fluid accumulation, and the gestational age. The size and unique specifications of the fetal shunt can then be determined based on the ultrasound. FIGS. 1A-B depict an illustration of one such device.

### Methods

Functionalities described in connection with the shunt(s) described herein are intended to be applicable to drainage of fluid with a shunt of the disclosure, e.g., treatment of a fetus(es) with excessive fluid in a region of their body, e.g., bladder shunting, thoracic shunting, or another type of shunting. Although fetal shunting is contemplated and exemplified, the disclosure contemplates the implementation of a shunt of FIG. 1 in other indications, e.g., urological and gastrointestinal indications. This shunting may allow life-saving drainage during development. It is specifically contemplated that a stent of the disclosure is used for draining fluid from accumulated spaces in certain fetal compartment(s), such as bladder in the lower urinary, and chest as in pleural effusion (unilateral or bilateral). There are many suitable implementations of the systems described herein. In many instances, the procedure is performed in an unborn fetus, although it is contemplated that the stents of the disclosure can also be used with premature infants.

In some aspects, the disclosure contemplates methods for treating a subject with shunts that have been manufactured according to the disclosure. In some aspects, the disclosure contemplates a shunt placement process that, using ultrasound or MRI guidance, inserts a needle through the mother's abdomen and uterus into the fetus's body where the fluid accumulation is present. A shunt of the disclosure is then placed to create a pathway for the fluid to drain from the body part of the subject (e.g., thoracic or bladder) into another cavity (e.g., the amniotic cavity).

Typically, both the mother and fetus are closely monitored during and after the procedure for any signs of complications. Regular follow-up appointments may be scheduled to monitor the shunt's position and function, as well as the overall health of the fetus and mother. The size of catheter (e.g., 3F, 4F, or 5F) can be selected based on the imaging of the fetus.

In some aspects, the disclosure provides a method for treating a fetus with excessive fluid in a region of its body, the method comprised of implanting into said fetus a fetal shunt comprising a hollow tube having an intermediary section, wherein said intermediary section does not have any perforations; a curved pigtail at a distal end of the hollow tube, wherein said distal curved pigtail comprises a plurality of perforations, said distal curved pigtail perpendicularly positioned with respect to said intermediary section; a curved pigtail at a proximal end of the hollow tube, wherein said proximal curved pigtail comprises a plurality of perforations, said proximal curved pigtail perpendicularly positioned with respect to said intermediary section; a barbed end placed between the intermediary section and the proximal end. In many aspects, the region of the fetus is a thoracic cavity or a bladder. In many instances, the implanting occurs on a fetus that is at least in gestational week 32, but the procedure may also occur on a fetus that is estimated to be on gestational week 28, gestational week 29, gestational week 30, gestational week 31, gestational week 32, gestational week 33, gestational week 34, gestational week 35, gestational week 36, gestational week 37, gestational week 38, gestational week 39, gestational week 40, or later. The implanting may occur on a fetus that is in-utero at any gestational week. The implanting may occur on a fetus that is born pre-maturely, i.e., before 40 weeks of gestation.

### EMBODIMENT

EMBODIMENT 1. A shunt comprising:
a hollow tube, wherein the hollow tube comprises two ends and an intermediary section, wherein the two ends are curved pigtails, and wherein one or more barbed ends are positioned between the intermediary section and at least one of the curved pigtails.

EMBODIMENT 2. A shunt comprising:
a hollow tube having an intermediary section;
a curved pigtail at a distal end of the hollow tube, wherein said distal curved pigtail comprises a plurality of perforations, said distal curved pigtail perpendicularly positioned with respect to said intermediary section;
a curved pigtail at a proximal end of the hollow tube, wherein said proximal curved pigtail comprises a plurality of perforations, said proximal curved pigtail perpendicularly positioned with respect to said intermediary section; and
one or more barbed ends placed between the intermediary section and the proximal end.

EMBODIMENT 3. The shunt of any one of embodiments 1 or 2, wherein the intermediary section has a length of between about 2.5 centimeters (cm) and about 4.5 cm.

EMBODIMENT 4. The shunt of any one of embodiments 1 or 2, wherein each of the two curved pigtails comprise one loop of at least 280 degrees.

EMBODIMENT 5. The shunt of any one of embodiments 1 or 2, wherein at least one of the two curved pigtails comprise a full loop and a half loop.

EMBODIMENT 6. The shunt of any one of embodiments 1 or 2, wherein one of the two curved pigtails has a clockwise orientation and the other has a counterclockwise orientation.

EMBODIMENT 7. The shunt of any one of embodiments 1 or 2, wherein each of the two curved pigtails have a same clockwise orientation.

EMBODIMENT 8. The shunt of any one of embodiments 1 or 2, wherein the shunt is a fetal shunt.

EMBODIMENT 9. The shunt of any one of embodiments 1 or 2, wherein one of the two curved pigtails is softer with respect to the rigidity of the intermediary segment.

EMBODIMENT 10. The shunt of any one of embodiments 1 or 2, wherein a tensile strength of one of the two curved pigtails is significantly softer than a tensile strength of the remaining of the shunt.

EMBODIMENT 11. The shunt of any one of embodiments 1 or 2, wherein said intermediary section does not have any perforations.

EMBODIMENT 12. The shunt of any one of embodiments 1 or 2, wherein the hollow tube has an unstretched length of about 2 cm to about 7 cm.

EMBODIMENT 13. The shunt of any one of embodiments 1 or 2, wherein the hollow tube has an unstretched length no longer than about 7 cm.

EMBODIMENT 14. The shunt of any one of embodiments 1 or 2, wherein the hollow tube has an unstretched length of about 4 cm.

EMBODIMENT 15. The shunt of any one of embodiments 1 or 2, wherein each of the two curved pigtails has a plurality of perforations.

EMBODIMENT 16. The shunt of Embodiment 15, wherein the plurality of perforations in at least one of the two curved pigtails is no more than 15 perforations.

EMBODIMENT 17. The shunt of Embodiment 15, wherein the plurality of perforations in at least one of the two curved pigtails is no more than 10 perforations.

EMBODIMENT 18. The shunt of Embodiment 15, wherein the plurality of perforations in both of the two curved pigtails is no more than 15 perforations.

EMBODIMENT 19. The shunt of Embodiment 15, wherein the plurality of perforations in both of the two curved pigtails is no more than 10 perforations.

EMBODIMENT 20. The shunt of any one of embodiments 1 or 2, wherein the outer diameter of the hollow tube is approximately 1 millimeter (mm) and its circumference is approximately 3.14 mm.

EMBODIMENT 21. The shunt of any one of embodiments 1 or 2, wherein the outer diameter of the hollow tube is approximately 1.33 mm and its circumference is approximately 4.19 mm.

EMBODIMENT 22. The shunt of any one of embodiments 1 or 2, wherein the outer diameter of the hollow tube is approximately 1.67 mm and its circumference is approximately 5.24 mm.

EMBODIMENT 23. The shunt of any one of embodiments 1 or 2, wherein the outer diameter of the hollow tube is approximately 2.00 mm and its circumference is approximately 6.28 mm.

EMBODIMENT 24. The shunt of any one of embodiments 1 or 2, wherein the one or more barbed ends positioned between the intermediary section and one of the curved pigtails are two barbed ends defining a proximal end of the shunt.

EMBODIMENT 25. The shunt of any one of embodiments 1 or 2, wherein the one or more barbed ends are two barbed ends symmetrically placed between the intermediary section and both of the curved pigtails.

EMBODIMENT 26. The shunt of any one of embodiments 1 or 2, wherein the one or more barbed ends comprise at least two barbed ends placed between the intermediary section and one or both of the curved pigtails.

EMBODIMENT 27. The shunt of any one of embodiments 1 or 2, wherein a tensile strength of the two curved pigtails is significantly softer than a tensile strength of the remaining of the shunt.

EMBODIMENT 28. A method for treating a subject with excessive fluid in a region of its body, the method comprised of implanting into said subject a shunt comprising a hollow tube, wherein the hollow tube comprises two ends and an intermediary section, wherein the two ends are curved pigtails, and wherein one or more barbed ends are positioned between the intermediary section and at least one of the curved pigtails.

EMBODIMENT 29. The method of Embodiment 28, wherein the region of the subject body is a bladder.

EMBODIMENT 30. The method of Embodiment 28, wherein the region of the subject body is a thoracic cavity.

EMBODIMENT 31. The method of Embodiment 28, wherein the implanting occurs on a subject that is in at least gestational week 32.

EMBODIMENT 32. The method of anyone of Embodiments 28-31, wherein the intermediary section has a length of between 2.5 cm and 4.5 cm.

EMBODIMENT 33. The method of anyone of Embodiments 28-31, wherein each of the two curved pigtails comprise one 360 degree full loop.

EMBODIMENT 34. The method of anyone of Embodiments 28-31, wherein at least one of the two curved pigtails comprise one and half loops.

EMBODIMENT 35. The method of anyone of Embodiments 28-34, wherein one of the two curved pigtails has clockwise orientation and the other has a counterclockwise orientation.

EMBODIMENT 36. The method of anyone of Embodiments 28-35, wherein each of the two curved pigtails have a same clockwise orientation.

EMBODIMENT 37. The method of anyone of Embodiments 28-36, wherein the shunt is a fetal shunt.

EMBODIMENT 38. The method of anyone of Embodiments 28-37, wherein the shunt comprises a softer pigtail at the proximal end with respect to the rigidity of the intermediary segment.

EMBODIMENT 39. The method of Embodiment 38, wherein a tensile strength of said proximal curved pigtail is softer than a tensile strength of said hollow tube.

EMBODIMENT 40. The method of anyone of Embodiments 28-39, wherein said intermediary section does not have any perforations.

EMBODIMENT 41. The method of anyone of Embodiments 28-40, wherein said hollow tube has a length of about 2 cm to 7 about cm.

EMBODIMENT 42. The method of anyone of Embodiments 28-41, wherein the hollow tube has a length no longer than 7 cm.

EMBODIMENT 43. The method of anyone of Embodiments 28-42, wherein the hollow tube has a length of about 4 cm.

EMBODIMENT 44. The method of anyone of Embodiments 28-43, wherein the plurality of perforations in at least one of the two curved pigtails is no more than 15 perforations.

EMBODIMENT 45. The method of anyone of Embodiments 28-44, wherein the plurality of perforations in at least one of the two curved pigtails is no more than 10 perforations.

EMBODIMENT 46. The method of anyone of Embodiments 28-45, wherein the plurality of perforations in at least one of the two curved pigtails is no more than 15 perforations.

EMBODIMENT 47. The method of anyone of Embodiments 28-45, wherein the plurality of perforations in at least one of the two curved pigtails is no more than 10 perforations.

EMBODIMENT 48. The method of anyone of Embodiments 28-45, wherein the outer diameter of the hollow tube is approximately 1 mm and its circumference is approximately 3.14 mm.

EMBODIMENT 49. The method of anyone of Embodiments 28-47, wherein the outer diameter of the hollow tube is approximately 1.33 mm and its circumference is approximately 4.19 mm.

EMBODIMENT 50. The method of anyone of Embodiments 28-47, wherein the outer diameter of the hollow tube is approximately 1.67 mm and its circumference is approximately 5.24 mm.

EMBODIMENT 51. The method of anyone of Embodiments 28-47, wherein the outer diameter of the hollow tube is approximately 2.00 mm and its circumference is approximately 6.28 mm.

EMBODIMENT 52. The method of anyone of Embodiments 28-51, wherein the one or more barbed ends positioned between the intermediary section and one of the curved pigtails are two barbed ends defining a proximal end of the shunt.

EMBODIMENT 53. The method of anyone of Embodiments 28-51, wherein the one or more barbed ends are two barbed ends symmetrically placed between the intermediary section and both of the curved pigtails.

EMBODIMENT 54. The method of anyone of Embodiments 28-51, wherein the one or more barbed ends comprise at least two barbed ends placed between the intermediary section and one or both of the curved pigtails.

EMBODIMENT 55. The method of anyone of Embodiments 28-51, wherein the shunt comprises a softer pigtail at the distal end with respect to the rigidity of the intermediary segment.

EMBODIMENT 56. The method of Embodiment 55, wherein a tensile strength of said distal curved pigtail is softer than a tensile strength of said hollow tube.

EMBODIMENT 57. The method of anyone of Embodiments 27-50, wherein the shunt comprises a softer pigtail at the proximal end with respect to the rigidity of the intermediary segment.

EMBODIMENT 58. The method of Embodiment 57, wherein a tensile strength of said distal curved pigtail is softer than a tensile strength of said hollow tube.

EMBODIMENT 59. The method of Embodiment 58, wherein the shunt comprises softer pigtails at both the proximal and distal end with respect to the rigidity of the intermediary segment.

EMBODIMENT 60. The method of Embodiment 59, wherein a tensile strength of said proximal and said distal curved pigtails is softer than a tensile strength of said hollow tube.

### Examples

### Example 1—Shunt of Embodiment 1

A first embodiment of a shunt contemplated by the disclosure is manufactured via 3D printing. Briefly, the 3D printing device is instructed to manufacture a device from a CAD model with the following specifications:

The CAD model describes two pigtails 101 and 103 3D printed with their open ends facing counter-clockwise from one another. The radius (r) of the pigtail is calculated from the specifications for the circumference (C) using the formula: [r=\frac{C}{2\pi}]. Given the circumference (C=1 cm), the radius of the pigtail is: approximately 0.16 cm. Thus, the CAD specifications for the radius of each pigtail with a 1 cm circumference are approximately 0.16 cm. Each pigtail comprises 18 holes placed at 90 degree angles from one another. Each pigtail is perpendicular to an intermediary section 102 of 2 cm in length. There are no shunting holes in the intermediary section. The CAD model also describes two barbs, placed at 180 degree angles with respect to one another. One barb faces towards the opening of the shunt of a distal pigtail, the other faces towards the opening of the shunt of a proximal pigtail. The CAD model dimensions specify a device to be inserted with a 3 Fr caterer: one where the outer diameter of the hollow tube is approximately 1 mm and its circumference is approximately 3.14 mm.

FIGS. 1A-B illustrate such shunt devices. As shown in FIG. 1A, the shunt has a hollow tube having an intermediary section, wherein said intermediary section does not have any perforations; a full curved pigtail having one 280 degree loop at a distal end of the hollow tube, wherein said distal curved pigtail comprises a plurality of perforations, said distal curved pigtail perpendicularly positioned with respect to said intermediary section; a curved pigtail at a proximal end of the hollow tube, wherein said proximal curved pigtail comprises a plurality of perforations, said proximal curved pigtail perpendicularly positioned with respect to said intermediary section; and a barbed end placed between the intermediary section and the proximal end. In this configuration, the barbed end defines the proximal end from the distal end. In different configurations, the distal end and the proximal end may be symmetrical.

### Example 2—Shunt of Embodiment 2

A second embodiment of a shunt contemplated by the disclosure was manufactured with a heat mold technique. FIG. 1B shows a shunt manufactured using a heat mold technique. Briefly, the device was heat molded to the following specifications. The shunt comprises a proximal loop 106, a distal loop 109, a mid segment 108 located between the proximal loop 106 and the distal loop 109, and barbs 107 located along the mid segment 108 located nearer to the proximal loop 106 than the distal loop 109. As shown in FIG. 1B, the proximal loop 106 and the distal loop 109 each comprise a double loop with a 90 degree angle to the mid section 108.

Although FIG. 1B depicts the proximal loop 106 and the distal loop 109 as each comprising a double loop, the disclosure is not intended to be so limiting. For instance, in some embodiments, the proximal loop 106 or the distal loop 109 each independently comprise a single loop, a double loop, a triple loop, a quadruple loop, and so forth. In some embodiments, the proximal loop 106 and the distal loop 109 comprise the same number of loops (e.g., the proximal loop 106 and the distal loop 109 each comprise a single loop, a double loop, a triple loop, a quadruple loop, or the like). In some embodiments, the proximal loop 106 and the distal loop 109 comprise a different number of loops (e.g. the proximal loop 106 comprise a single loop and the distal loop 109 comprises a double loop).

Further, although FIG. 1B depicts the proximal loop 106 and the distal loop 109 as each being at a 90 degree angle to the mid section 108, the disclosure is not intended to be so limiting. For instance, in some embodiments, the proximal loop 106 or the distal loop 109 are at non-90 degree angles (such as approximately 5 degree angles, 10 degree angles, 15 degree angles, 20 degree angles, 25 degree angles, 30 degree angles, 35 degree angles, 40 degree angles, 45 degree angles, 50 degree angles, 55 degree angles, 60 degree angles, 65 degree angles, 70 degree angles, 75 degree angles, 80 degree angles, 85 degree angles, and so forth) to the mid section 108. In some embodiments, the proximal loop 106 and the distal loop 109 are at substantially equal angles to the mid section 108. In some embodiments, the proximal loop 106 and the distal loop 109 are at different angles to the mid section 108.

Further still, although FIG. 1B depicts the barbs 107 as being located nearer to the proximal loop 106 than the distal loop 109, the disclosure is not intended to be so limiting. For instance, in some embodiments, the barbs 107 may be located farther from the proximal loop 106 than the distal loop 109. Alternatively, the barbs 107 may be located substantially equidistant from the proximal loop 106 and the distal loop 109.

### Example 3—Animal Studies

Intrauterine fetal shunts have been used to drain obstructed or accumulated fluid from fetal organs, which, if left untreated, could lead to severe health risks and even mortality. Fetal shunts are generally manufactured as flexible plastic tubes, placed in utero under continuous ultrasound monitoring, guided percutaneously to a fetal space that has fluid accumulation. Vesicoamniotic shunts for lower urinary tract obstruction and thoracoamniotic shunts for fetal hydrothorax are the two most common indications for shunt placement. The current FDA approved shunt is the Harrison Fetal Bladder Stent set (Cook Medical Inc., Bloomington, IN, USA)) in the United States as a Humanitarian Use Device (Humanitarian Device Exemption HDE #H960001 for the treatment of lower urinary tract obstruction (LUTO) decompression following the diagnosis of fetal post-vesicular obstructive uropathy in fetuses at 18 to 32 weeks gestational age. This shunt is also used off-label as a thoracoamniotic shunt. There is a 60-80% risk of shunt dislodgement and many fetuses undergo 2 or more shunt placements; with the rate of dislodgement higher in earlier gestation. Placing these pregnancies at risk for preterm labor, preterm birth, membrane separation and preterm premature rupture of membranes (PPROM). Currently there are no other FDA approved alternatives to Harrison Fetal Bladder stent.

The present disclosure sought to compare alternative devices to existing stents, with specifications that would facilitate anchoring and reduce dislodgement as readily.

Rats with pleural effusion are created as animal models. Bilateral pleural effusions are created in approximately 5 euthanized rat subjects. Using ultrasound guidance, a 21-gauge butterfly intravenous (IV) set needle is introduced into the T7 pleural space in each rat model under ultrasound guidance. The pleural effusion is created followed by placement of a stent of Example 1 onside, followed by a comparator stent in the contralateral size in sequence.

Weighted binder clips are attached to the end of the stent with serial attachment of weights to assess the maximum traction force the stents could endure. During the progressive attachments of the weights to the end of the stents, the maximum weight to cause stent displacement and dislodgement is recorded. Direct comparison of dislodgement weight of are logged.

Following completion of the animal model, this technique can be used to implement a similar stent in a subject.

### Example 4—Evaluation of Dislocation, Dislodgement, and Occlusion of Various Stents in Rat Models

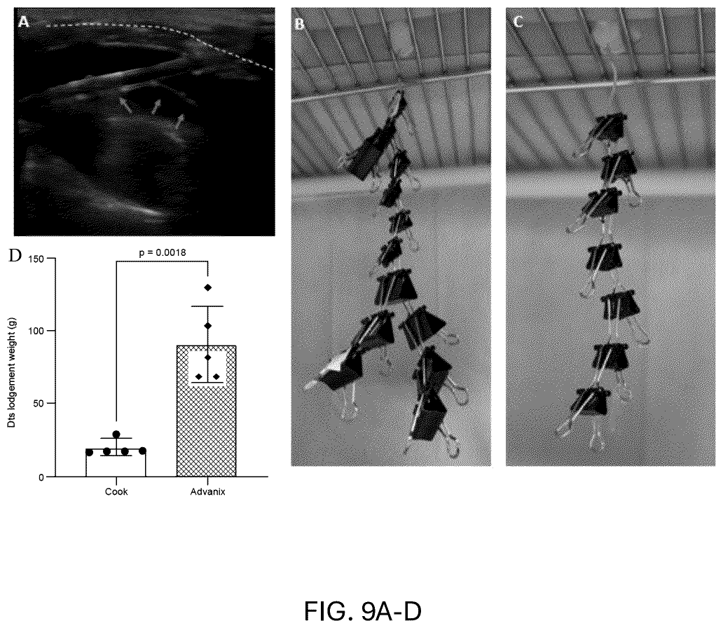

Fetal LUTO and hydrothorax are common indications for shunt placement in utero. We conducted experiments comparing the stents depicted in FIG. 2 and 3D in five 275±20 g Sprague Dawley nude rats under an Institutional Animal Care and Use Committee (IACUC) exemption See FIGS. 9A-D. Following euthanasia, under ultrasound guidance, a needle was inserted into the pleural space under the T7 level, and 20 cc of saline was injected to induce a pleural effusion FIG. 9A. Both shunts were deployed in contralateral pleural spaces at the T7-T8 level. The distal ends were secured with a clip to a suture, allowing the attachment of weights to assess the maximum traction force the stents could endure. See FIGS. 9B-C. The procedure was replicated on the contralateral pleural cavity using a stent depicted in FIG. 3D. The stents and shunts were noted to be draining the chest in all experiments. The mean dislodgement weight was 18.88±5.58 g for the weight depicted in FIG. 2; and the mean dislodgement weight was 90.14±26.21 g (p=0.002) for the weight depicted in FIG. 3D. FIG. 9D. The distal end was secured with a clip to a suture, allowing the attachment of weights (FIG. 9B). The procedure was replicated on the contralateral pleural cavity using a stent as shown in FIG. 3D (See FIG. 3D; FIG. 9C). The sides and types attempted were randomly selected. Weighted binder clips were attached to the end of the stent, then additional weights were attached serially to determine the maximum traction force the stents could endure. The maximum weights needed to dislodge each stent were recorded and compared (FIG. 9D). The mean dislodgement weight was HFBS and 90.14±26.21 g for the stent as shown in FIG. 3D vs FIG. 2 devices. Table 1 compares dislodgement weights between shunts in each animal.

TABLE 1

| | | Left | Right | Stent of | Stent of |
|---|---|---|---|---|---|
| | Rat | pleural | pleural | FIG. 2 | FIG. 3 |
| | weight | effusion | effusion | weight | Dislodgement |
| | (g) | (cc) | (cc) | (g) | weight (g) |
| Rat # 1 | 250 | 25 | 25 | 14.3 | 68.26 |
| Rat # 2 | 270 | 30 | 25 | 17.16 | 68.26 |
| Rat # 3 | 412 | 35 | 35 | 28.6 | 129.42 |
| Rat # 4 | 301 | 35 | 30 | 17.16 | 81.48 |
| Rat # 5 | 245 | 35 | 35 | 17.16 | 103.28 |

Ex-vivo testing of dislodgement rates in weights (g) comparing stents on rat models with pleural effusions

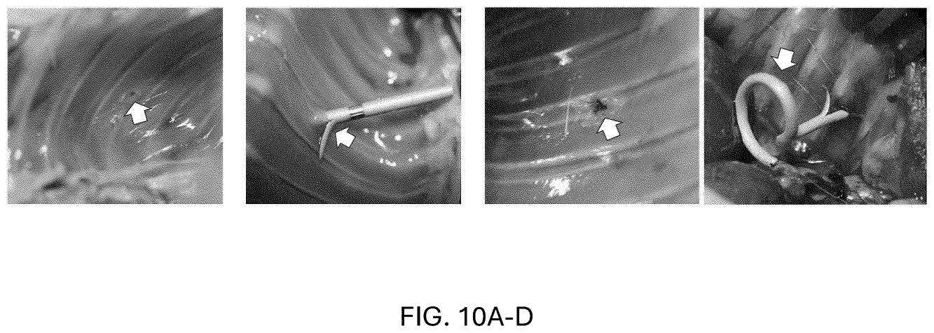

We also evaluated the site of entry of certain stents. FIG. 10A depicts the stent entry site of a stent with a design shown in FIG. 2. FIG. 10B depicts the functional utility of the barb of a stent with a design shown in FIG. 2 in preventing dislodgement. FIG. 10C depicts the stent entry site of a stent with a design shown in FIG. 3A of size 5Fr.

### Example 5—Evaluation of Various Stents in Humans

After completing the animal studies and obtaining fetal ethics approval and informed consent, we consider the use of a stent comprising a barb wire and a pig tail in a patient with a fetal diagnosis of LUTO at 14 weeks gestation for whom two stents of the configuration shown in FIG. 2 had already failed. Given the risk of poor neonatal outcomes, including mortality secondary to pulmonary hypoplasia, if no additional shunt were placed, we requested approval from the Fetal Innovation and Ethics Therapy Board (FIETB) at UTHealth Houston to use the commercially available stent with the design depicted in FIG. 3D pancreatic stent off label in the case of the 28-year-old woman.

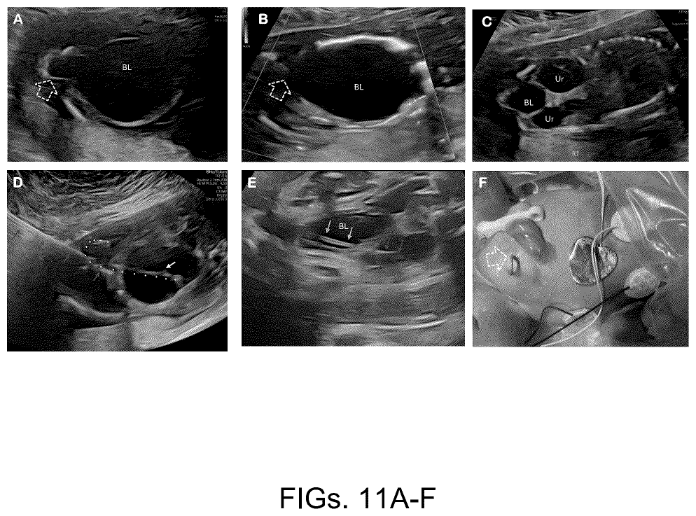

The 28-year-old pregnant woman, Gravida 1 Para 0, was diagnosed with LUTO at 14 weeks gestation. See FIG. 11A. Chorionic villous sampling and fetal vesicocentesis (bladder drainage) showed a normal fetal karyotype, chromosomal microarray and fetal renal function. As previously mentioned, the patient had a stent with no barb (FIG. 2 configuration) placed at 17 weeks that dislodged 7 days later. A second stent of the same design (no barb, FIG. 2 configuration) was placed at 19 weeks and dislodged 24 hours later. The stents depicted in FIG. 3D are commercially available in several sizes, including a 5 Fr stent that fits through a 13 G needle. Although these stents fail to have the second pig tail depicted in FIG. 1, we proceeded to implement it in the patient to consider a potential dislodgement improvement offered by the barb wire with one pig coil. This was possible due to the commercial availability of the stent in FIG. 3D.

At 22 weeks' gestation, a stent of FIG. 3D was placed similarly to the stent of FIG. 2 by using a 13 G needle with the stent advanced over a guide wire. The barb at the outer end and the stent were released by retracting the insertion cannula from the fetal bladder; the pigtail subsequently rested on the fetal bladder. In our case, we noticed that the barbed catheter prevented the dislodgement of the stent. See FIG. 11B. FIG. 11C is an ultrasound depicting the patient at 22 weeks with bilateral hydroutereter and hydronephrosis. FIG. 11D is an ultrasound depicting the patient at 22 weeks 3 days during the placement of stent (5 Fr; configuration of FIG. 3D) 5 cm in length in the bladder. Cook needle and introducer noted through the maternal abdominal wall and uterus (blue arrows), and stent barbed (yellow arrow) and pigtail noted outside the fetal abdomen. See FIG. 11D.

During the procedure, it was confirmed the bladder had collapsed (not shown). Spontaneous renal failure with onset of oligohydramnios occurred at 26 weeks. After an additional FIETB approval the patient underwent serial amnioinfusions to improve pulmonary growth and development. We noted that the stent pushed the bladder wall further into the abdomen at 27 weeks.

FIG. 11E is an ultrasound depicting the patient at 30 weeks showing the stent in place through the bladder (white arrow) and piercing through the bladder dome into the peritoneal space with collection of fluid (red arrow).

Premature rupture of membranes occurred at 32 weeks, and the patient delivered a viable male fetus. On day of life (DOL) 2, the infant was taken to the operating room by pediatric urology where the stent was removed and noted to be into the abdomen and folded over into the bladder. FIG. 11F is a photograph depicting a stent of configuration 3d coming through neonatal abdominal wall (white dashed arrow) at birth. Renal ultrasound at birth confirmed bilateral renal hypoplasia and decreased corticomedullary differentiation. The infant was admitted to the neonatal intensive care unit (ICU) and discharged home on DOL 126 on peritoneal dialysis, gastrostomy tube for feeding, and home oxygen.

At conclusion, the barb at the outer end and the stent was released by retracting the insertion cannula from the fetal bladder; the single pigtail subsequently rested on the fetal bladder. It was observed that the barbed catheter prevented the stent from dislodging.

The present technology is not to be limited in terms of the particular implementations described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular implementations only, and is not intended to be limiting.

1. (canceled)

2. A shunt comprising:
a hollow tube having an intermediary section;
a curved pigtail at a distal end of the hollow tube, wherein said distal curved pigtail comprises a plurality of perforations, said distal curved pigtail perpendicularly positioned with respect to said intermediary section;
a curved pigtail at a proximal end of the hollow tube, wherein said proximal curved pigtail comprises a plurality of perforations, said proximal curved pigtail perpendicularly positioned with respect to said intermediary section; and
one or more barbed ends placed between the intermediary section and the proximal end.

3. The shunt of claim 2, wherein the intermediary section has a length of between about 2.5 centimeters (cm) and about 4.5 cm.

4. The shunt of claim 2, wherein each of the two curved pigtails comprise one loop of at least 280 degrees.

5. The shunt of claim 2, wherein at least one of the two curved pigtails comprise a full loop and a half loop.

6. The shunt of claim 2, wherein one of the two curved pigtails has a clockwise orientation and the other has a counterclockwise orientation.

7. The shunt of claim 2, wherein each of the two curved pigtails have a same clockwise orientation.

8. The shunt of claim 2, wherein the shunt is a fetal shunt, a GI shunt, or a urology shunt.

9. (canceled)

10. The shunt of claim 2, wherein a tensile strength of one of the two curved pigtails is significantly softer than a tensile strength of the remaining of the shunt.

11. The shunt of claim 2, wherein said intermediary section does not have any perforations.

12. The shunt of claim 2, wherein the hollow tube has an unstretched length no longer than about 7 cm.

13.-14. (canceled)

15. The shunt of claim 2, wherein each of the two curved pigtails has a plurality of perforations.

16-23. (canceled)

24. The shunt of claim 2, wherein the one or more barbed ends positioned between the intermediary section and one of the curved pigtails are two barbed ends defining a proximal end of the shunt.

25. The shunt of claim 2, wherein the one or more barbed ends are two barbed ends symmetrically placed between the intermediary section and both of the curved pigtails.

26. The shunt of claim 2, wherein the one or more barbed ends comprise at least two barbed ends placed between the intermediary section and one or both of the curved pigtails.

27. The shunt of claim 2, wherein a tensile strength of the two curved pigtails is significantly softer than a tensile strength of the remaining of the shunt.

28-60. (canceled)

61. The shunt of claim 10, wherein the softer curved pigtail is at a proximal end, and wherein the softer proximal pigtail prevents organ damage.

62. The shunt of claim 61, wherein the softer curved pigtail at a distal end remains stiffer, and wherein the stiffer distal pigtail reduces dislodgement into the body.

63. The shunt of claim 24, wherein the barbed ends at the proximal end facilitate the anchoring of the shunt in place, easing the placement and decreasing the dislodgement, while the curved pigtail at the distal end facilitates the maintenance of the shunt in place.

64. The shunt of claim 10, wherein the softer curved pigtail is at a distal end, and wherein the softer distal pigtail reduces the risk of entanglement by hand or finger movement from the fetus.
